SEQUENCE LISTING (42 sequences)

SEQ ID NO: 1 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 2 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 3 - protein, 100 aa, Artificial Sequence
  Description of Artificial Sequence Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 4 - DNA, 1539 bp, Homo sapiens
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 5 - DNA, 345 bp, Homo sapiens
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 6 - protein, 115 aa, Homo sapiens; identical to SEQ ID NO: 2

SEQ ID NO: 7 - protein, 115 aa, Ovis aries
ILGRSETQECIFYNANWERDRTNRTGVESCYGDKDKRRHCFATWKNISGSIDIVKQGCWLDDINCYDRTDCIEKKDSPEVYFCCCEGNMCNERFSYFPEMEVTQPTSNPVTPKPP

SEQ ID NO: 8 - protein, 115 aa, Condylura cristata
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 9 - protein, 115 aa, Mus musculus
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 10 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 11 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 3 aa, Artificial Sequence
  Description of Artificial Sequence Synthetic peptide
GGG

SEQ ID NO: 17 - protein, 4 aa, Artificial Sequence
  Description of Artificial Sequence Synthetic peptide
GGGG

SEQ ID NO: 18 - protein, 5 aa, Artificial Sequence
  Description of Artificial Sequence Synthetic peptide
TGGGG

SEQ ID NO: 19 - protein, 5 aa, Artificial Sequence
  Description of Artificial Sequence Synthetic peptide
SGGGG

SEQ ID NO: 20 - protein, 4 aa, Artificial Sequence
  Description of Artificial Sequence Synthetic peptide
TGGG

SEQ ID NO: 21 - protein, 4 aa, Artificial Sequence
  Description of Artificial Sequence Synthetic peptide
SGGG

SEQ ID NO: 22 - protein, 5 aa, Artificial Sequence
  Description of Artificial Sequence Synthetic peptide
GGGGS

SEQ ID NO: 23 - protein, 344 aa, Artificial Sequence
  Description of Artificial Sequence Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 21 aa, Artificial Sequence
  Description of Artificial Sequence Synthetic peptide
MKFLVNVALVFMVVYISYIYA

SEQ ID NO: 25 - protein, 22 aa, Artificial Sequence
  Description of Artificial Sequence Synthetic peptide
MDAMKRGLCCVLLLCGAVFVSP

SEQ ID NO: 26 - protein, 20 aa, Artificial Sequence
  Description of Artificial Sequence Synthetic peptide
MGAAAKLAFAVFLISCSSGA

SEQ ID NO: 27 - protein, 369 aa, Artificial Sequence
  Description of Artificial Sequence Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1114 bp, Artificial Sequence
  Description of Artificial Sequence Synthetic polynucleotide
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 29 - protein, 9 aa, Artificial Sequence
  Description of Artificial Sequence Synthetic peptide
ILGRSETQE

SEQ ID NO: 30 - protein, 329 aa, Artificial Sequence
  Description of Artificial Sequence Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 32 - protein, 225 aa, Artificial Sequence; identical to SEQ ID NO: 11
  Description of Artificial Sequence Synthetic polypeptide

SEQ ID NO: 33 - protein, 229 aa, Artificial Sequence; identical to SEQ ID NO: 15
  Description of Artificial Sequence Synthetic polypeptide

SEQ ID NO: 34 - protein, 223 aa, Artificial Sequence; identical to SEQ ID NO: 12
  Description of Artificial Sequence Synthetic polypeptide

SEQ ID NO: 35 - protein, 232 aa, Artificial Sequence; identical to SEQ ID NO: 13
  Description of Artificial Sequence Synthetic polypeptide

SEQ ID NO: 36 - protein, 115 aa, Bos taurus
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 37 - protein, 115 aa, Tyto alba; identical to SEQ ID NO: 10

SEQ ID NO: 38 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 39 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 40 - protein, 100 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 41 - protein, 343 aa, Artificial Sequence
  Description of Artificial Sequence Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 6 aa, Artificial Sequence
  Description of Artificial Sequence Synthetic 6xHis tag
HHHHHH

(19) United States
(12) Patent Application Publication
(10) Pub. No.: US 2024/0174733 A1
(43) Pub. Date: May 30, 2024

(54) ACTRII PROTEINS AND USES THEREOF

(71) Applicant: Acceleron Pharma Inc., Cambridge, MA (US)

(72) Inventors: Gang Li, Cambridge, MA (US); Ravindra Kumar, Cambridge, MA (US)

(21) Appl. No.: 18/280,899

(22) PCT Filed: Mar. 9, 2022
(86) PCT No.: PCT/US2022/019583

§ 371 Date: Sep. 7, 2023

Related U.S. Application Data
(60) Provisional application No. 63/159,074, filed on Mar. 10, 2021.

(51) Int. Cl. C07K 14/71; A61P 3/10

(52) U.S. Cl. CPC C07K 14/71; A61P 3/10; C07K 2319/30

(57) ABSTRACT

In some aspects, the disclosure relates to compositions and methods comprising ActRII polypeptides to treat, prevent, or reduce the progression rate and/or severity of hyperglycemia, particularly treating, preventing or reducing the progression rate and/or severity of one or more hyperglycemia-associated complications.

### CROSS-REFERENCE TO RELATED APPLICATIONS

This application claims the benefit of priority from U.S. Provisional Application No. 63/159,074, filed Mar. 10, 2021. The specification of the foregoing application is incorporated herein by reference in its entirety.

### BACKGROUND OF THE INVENTION

Hyperglycemia is a disease characterized by a dysregulation in glucose homeostasis which results in a subject having higher than normal blood glucose levels. Some of the factors that contribute to hyperglycemia include reduced insulin secretion, decreased glucose utilization, and increased glucose production.

Insulin is the most important regulator of glucose homeostasis as it stimulates the utilization of dietary glucose by peripheral tissues while also repressing hepatic glucose production. Decreased insulin production and/or reduced insulin sensitivity are important contributing factors to the development of hyperglycemia and they represent the underlying abnormalities of diabetes. In addition to decreased insulin secretion, diabetes is also characterized by impaired glucagon production, which can predispose to the risk of hypoglycemia in these patients.

The incidence of hyperglycemia has increased dramatically over the last two decades due to increased obesity, decreased activity level, and an aging population. The consequences of hyperglycemia have been associated with comorbidities including pre-diabetes mellitus, type 1 diabetes mellitus, type 2 diabetes mellitus, complications associated with diabetes mellitus, cataracts, microvascular disease, macrovascular disease, nephropathy, diabetic nephropathy, glomerulosclerosis, retinopathy, diabetic retinopathy, choroidal neovascularisation, coronary artery disease, cerebrovascular disease, peripheral vascular disease, diabetic ketoacidosis (DKA), hyperosmolar hyperglycemie state (HHS), non-alcoholic fatty liver (NAFL) disease, non-alcoholic steatohepatitis (NASH), neuropathy, diabetic neuropathy, diabetic pain, tissue ischaemia, diabetic foot, diabetic ulcer, arteriosclerosis, hepatic steatosis, hypertension, and polycystic ovary syndrome.

Current therapeutic approaches to hyperglycemia include major modifications in an individual's dietary nutrition, increasing physical activity, anti-hyperglycemic drugs, and insulin. However, hyperglycemia is chronic and progressive, and, to date, no treatment is able to reverse the progression. There is a high, unmet need for effective therapies for treating hyperglycemic conditions, such as insulin resistance, impaired glucose tolerance, diabetes, and hypertension in a patient, and related conditions. Accordingly, it is an object of the present disclosure to provide methods for treating, preventing, or reducing the progression rate and/or severity of hyperglycemic conditions, particularly treating, preventing or reducing the progression rate and/or severity of one or more complications of hyperglycemia.

### SUMMARY OF THE INVENTION

In some aspects, the disclosure provides a method of treating hyperglycemia associated with pulmonary hypertension, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of treating, preventing, or reducing the progression rate and/or severity of one or more complications of hyperglycemia associated with pulmonary hypertension, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the one or more complications of hyperglycemia associated with pulmonary hypertension is a complication selected from the group consisting of pre-diabetes mellitus, type 1 diabetes mellitus, type 2 diabetes mellitus, complications associated with diabetes mellitus, cataracts, microvascular disease, macrovascular disease, nephropathy, diabetic nephropathy, glomerulosclerosis, retinopathy, diabetic retinopathy, choroidal neovascularisation, coronary artery disease, cerebrovascular disease, peripheral vascular disease, DKA, HHS, NAFL disease, NASH, neuropathy, diabetic neuropathy, diabetic pain, tissue ischaemia, diabetic foot, diabetic ulcer, arteriosclerosis, hepatic steatosis, hypertension, and polycystic ovary syndrome.

In some aspects, the disclosure provides a method of treating hyperglycemia, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of treating, preventing, or reducing the progression rate and/or severity of one or more complications of hyperglycemia, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the one or more complications of hyperglycemia is selected from the group consisting of pre-diabetes mellitus, type 1 diabetes mellitus, type 2 diabetes mellitus, complications associated with diabetes mellitus, cataracts, microvascular disease, macrovascular disease, nephropathy, diabetic nephropathy, glomerulosclerosis, retinopathy, diabetic retinopathy, choroidal neovascularisation, coronary artery disease, cerebrovascular disease, peripheral vascular disease, DKA, HHS, NAFL disease, NASH, neuropathy, diabetic neuropathy, diabetic pain, tissue ischaemia, diabetic foot, diabetic ulcer, arteriosclerosis, hepatic steatosis, hypertension, and polycystic ovary syndrome.

In some embodiments, the patient further has pulmonary hypertension. In some embodiments, the pulmonary hypertension is combined post- and pre-capillary hypertension (CpcPH). In some embodiments, the pulmonary hypertension is Group 2 pulmonary hypertension. In some embodiments, the administration of the polypeptide results in a decrease in fasting glucose levels in the patient to levels less than 125 mg/dL. In some embodiments, the administration of the polypeptide results in a decrease in fasting glucose levels in the patient to levels less than 100 mg/dL. In some embodiments, the administration of the polypeptide results in a decrease in fasting glucose levels in the patient by at least 15%. In some embodiments, the administration of the polypeptide decreases blood glucose levels two hours after eating by at least 15% as compared to an untreated control. In some embodiments, the administration of the polypeptide decreases blood glucose levels two hours after eating by at least 50 mg/dL as compared to an untreated control. In some embodiments, the administration of the polypeptide decreases the glucose/creatinine ratio in the patient's urine to less than 15 mg/mg. In some embodiments, the administration of the polypeptide decreases the glucose/creatinine ratio in the patient's urine by at least 50% as compared to an untreated control. In some embodiments, the administration of the polypeptide decreases the glucose/creatinine ratio in the patient's urine by at least 30 mg/mg.

In some aspects, the disclosure provides a method of treating hyperglycemia in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, thereby decreasing fasting glucose levels in the patient. In some embodiments, the fasting glucose levels in the patient are decreased to levels less than 125 mg/dL. In some embodiments, the fasting glucose levels in the patient are decreased to levels less than 100 mg/dL. In some embodiments, the fasting glucose levels in the patient are decreased by at least 15%.

In some aspects, the disclosure provides a method of treating hyperglycemia in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, thereby decreasing blood glucose levels two hours after eating in the patient. In some embodiments, the blood glucose levels two hours after eating are decreased by at least 15%. In some embodiments, the blood glucose levels two hours after eating are decreased by at least 50 mg/dL.

In some aspects, the disclosure provides a method of treating hyperglycemia in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, thereby decreasing the glucose/creatine ratio in the patient's urine. In some embodiments, the glucose/creatine ratio in the patient's urine is decreased to less than 15 mg/mg. In some embodiments, the glucose/creatine ratio in the patient's urine is decreased by at least 50% as compared to an untreated control. In some embodiments, the glucose/creatine ratio in the patient's urine is decreased by at least 30 mg/mg as compared to an untreated control.

In some aspects, the disclosure provides a method of increasing insulin sensitivity in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some aspects, the disclosure provides a method of treating insulin resistance in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of increasing glucose utilization in a patient in need thereof, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of treating a glucose metabolism disease in a patient in need thereof, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the glucose metabolism disease is selected from the group consisting of glucose intolerance, insulin resistance, impaired glucose tolerance (IGT), and impaired fasting glucose.

In some aspects, the disclosure provides a method of treating hyperlipidemia comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some aspects, the disclosure provides a method of treating dyslipidemia comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of treating arteriosclerosis comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at anyone of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of reducing atherosclerotic plaque size in a subject in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of treating non-alcoholic fatty liver disease (NAFLD) comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the NAFLD is NASH.

In some aspects, the disclosure provides a method of treating non-alcoholic steatohepatitis (NASH) in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the subject exhibits one or more symptoms of NASH selected from the group consisting of weakness, fatigue, unexplained weight loss, ache and jaundice.

In some aspects, the disclosure provides a method of treating fibrosis in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of treating a fibrosis associated disorder in a patient in need thereof, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the fibrosis associated disorder is selected from the group consisting of pulmonary fibrosis, cardiac fibrosis, hypersensitivity pneumonitis, idiopathic fibrosis, tuberculosis, pneumonia, cystic fibrosis, asthma, chronic obstructive pulmonary disease (COPD), emphysema, renal (kidney) fibrosis, renal failure, chronic renal disease, bone fibrosis, myelofibrosis, rheumatoid arthritis, systemic lupus erythematosus, scleroderma, sarcoidosis, granulomatosis with polyangiitis, Peyronie's disease, liver fibrosis, Wilson's disease, glycogen storage diseases (particularly types III, IV, IX, and X), iron-overload, Gaucher disease, Zellweger syndrome, nonalcoholic and alcoholic steatohepatitis, biliary cirrhosis, sclerosing cholangitis, Budd-Chiari syndrome, surgery-associated fibrosis, Crohn's disease, Duputren's contracture, mediastinal fibrosis, nephrogeneic fibrosis, retroperitoneal fibrosis, atrial fibrosis, endomyocardial fibrosis, and pancreatic fibrosis. In some embodiments, the patient further has a condition selected from the group consisting of prediabetes, diabetes mellitus, type 1 diabetes mellitus, and type 2 diabetes mellitus. In some embodiments, the patient further has pulmonary hypertension. In some embodiments, the patient has Group 2 pulmonary hypertension. In some embodiments, the patient has CpcPH. In some embodiments, the patient has pulmonary arterial hypertension.

In some aspects, the disclosure provides a method of treating diabetes comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some aspects, the disclosure provides a method of treating, preventing, or reducing the progression rate and/or severity of one or more complications of diabetes, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the one or more complications of diabetes is selected from the group consisting of DKA, vascular complications, hypertension, myocardial infarction, peripheral artery disease, vasoregulation dysfunction, arteriosclerosis, microvascular damage (e.g., retinopathy, nephropathy, and neuropathy), macrovascular damage, peripheral neuropathy, decreased vision, cataracts, blindness, cardiovascular disease, autonomic nervous system damage (e.g., decreased heart rate variability), liver disease, fatty liver disease, steatohepatitis, steatosis, cirrhosis, renal disease, and end-stage renal disease.

In some aspects, the disclosure provides a method of treating, preventing, or reducing the progression rate and/or severity of one or more diabetes associated disorders, comprising administering to a patient in need thereof an effective amount of an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the one or more diabetes associated disorders is selected from the group consisting of dyslipidemia, hyperlipidemia, hypercholesterolemia, low HDL serum level, high LDL serum level, hypertriglyceridemia, IGT, maturity-onset diabetes of youth, leprechaunism, tropical diabetes, diabetes secondary to a pancreatic disease or surgery, diabetes associated with a genetic syndrome, pancreatitis, diabetes secondary to endocrinopathies, and adipositas. In some embodiments, the one or more complications of diabetes comprises one or more of the complications selected from prolonged wound healing, peripheral neurosenstivity, cataracts, micro- and macrovascular diseases, such as diabetic nephropathy, glomerulosclerosis, diabetic retinopathy, choroidal neovascularisation, NAFL disease, NASH, diabetic neuropathy, diabetic pain, tissue ischaemia, diabetic foot, diabetic ulcer, arteriosclerosis, myocardial infarction, acute coronary syndrome, unstable angina pectoris, stable angina pectoris, stroke, peripheral arterial occlusive disease, cardiomyopathy, heart failure, cardiovascular death, heart rhythm disorders and vascular restenosis. In some embodiments, the patient has decreased renal function. In some embodiments, the method further improves renal function. In some embodiments, the patient further has pulmonary hypertension. In some embodiments, the pulmonary hypertension is pulmonary arterial hypertension. In some embodiments, the pulmonary hypertension is post-capillary pulmonary hypertension (PcPH). In some embodiments, the PcPH is isolated post-capillary pulmonary hypertension (IpcPH). In some embodiments, the PcPH is combined post- and pre-capillary PH (CpcPH). In some embodiments, the pulmonary hypertension is Group 2 pulmonary hypertension.

In some embodiments of any of the methods disclosed herein, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of amino acids corresponding to residues 30-110 of SEQ ID NO: 1. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence SEQ ID NO: 2. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In some embodiments, the ActRII polypeptide is a fusion protein further comprising an Fc domain of an immunoglobulin. In some embodiments, the Fc domain is positioned at the N-terminus of the ActRII polypeptide. In other embodiments, the Fc domain is positioned at the C-terminus of the ActRII polypeptide. In some embodiments, the Fc domain of the immunoglobulin is an Fc domain of an IgG1 immunoglobulin. In some embodiments, the Fc fusion protein further comprises a linker domain positioned between the ActRII polypeptide domain and the Fc domain of the immunoglobulin. In some embodiments, the linker domain is selected from the group consisting of: TGGG (SEQ ID NO: 20), TGGGG (SEQ ID NO: 18), SGGGG (SEQ ID NO: 22), GGGGS (SEQ ID NO: 22), GGG (SEQ ID NO: 16), GGGG (SEQ ID NO: 17), and SGGG (SEQ ID NO: 21). In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41. In some embodiments, the polypeptide comprises an amino acid sequence that is at least 90% identical to an amino acid sequence corresponding to residues 30-110 of SEQ ID NO: 1, wherein the polypeptide binds to activin and/or GDF11. In some embodiments, the polypeptide comprises an amino acid sequence that is at least 90% identical to an amino acid sequence corresponding to residues 21-135 of SEQ ID NO: 1, wherein the polypeptide binds to activin and/or GDF11.

In some embodiments of any of the methods disclosed herein, the polypeptide is lyophilized. In some embodiments, the polypeptide is soluble. In some embodiments of any of the methods disclosed herein, the polypeptide is administered using subcutaneous injection. In some embodiments, the polypeptide is administered every 3 weeks. In some embodiments, the polypeptide is administered every 4 weeks. In some embodiments, the polypeptide is part of a homodimer protein complex. In some embodiments, the polypeptide is glycosylated. In some embodiments, the polypeptide has a glycosylation pattern obtainable by expression in a Chinese hamster ovary cell. In some embodiments, the ActRII polypeptide binds to one or more ligands selected from the group consisting of: activin A, activin B, and GDF11. In some embodiments, the ActRII polypeptide further binds to one or more ligands selected from the group consisting of: BMP10, GDF8, and BMP6. In some embodiments, the ActRII polypeptide is administered at a dose between 0.1 mg/kg and 2.0 mg/kg. In some embodiments, the ActRII polypeptide is administered at a dose of 0.3 mg/kg. In some embodiments, the ActRII polypeptide is administered at a dose of 0.7 mg/kg.

In some embodiments of any of the methods disclosed herein, the method further comprises administering to the patient an additional active agent and/or supportive therapy. In some embodiments, the additional active agent and/or supportive therapy is selected from the group consisting of: a phosphodiesterase (PDE) inhibitor, sirtuin pathway activator, anti-diabetic agent, anti-hyperlipidemic agent, glucagon-like peptide-1 (GLP-1) agonist, dipeptidyl peptidase-4 (DPP-4) inhibitor, sodium-glucose cotransporter-2 (SGLT2) inhibitor or gliflozin, a fibrate, a nitric oxide (NO) donor, nicotinic acid, nicotinamide riboside, nicotinic acid metabolite, and any combination thereof. In some embodiments, the phosphodiesterase (PDE) inhibitor is a phosphodiesterase type 5 (PDE5) inhibitor. In some embodiments, the PDE5 inhibitor is selected from the group consisting of avanafil, iodenafil, mirodenafil, sildenafil, tadalafil, icariin, vardenafil, udenafil, zaprinst, benzamidenafil, dasantafil, and any combination thereof. In some embodiments, the sirtuin pathway activator is selected from the group consisting of biguanide, resveratrol, a sirtuin activator, an AMPK activator, and a PGC-1a activator. In some embodiments, the PGC-1a activator is a thiazolidinedione. In some embodiments, the thiazolidinedione is selected from the group consisting of rosiglitazone, pioglitazone, lobeglitazone, and any combination thereof. In some embodiments, the anti-diabetic agent is selected from the group consisting of biguanide, meglitinide, sulfonylurea, thiazolidinedione, alpha glucosidase inhibitor, ergot alkaloid, and any combination thereof. In some embodiments, the sulfonylurea is glipizide. In some embodiments, the biguanide is metformin. In some embodiments, the glucagon-like peptide-1 (GLP-1) agonist is selected from the group consisting of exenatide, liraglutide, lixisenatide, albiglutide, dulaglutide, taspoglutide, semaglutide, and any combination thereof. In some embodiments, the iodipeptidyl peptidase-4 (DPP-4) inhibitor is selected from the group consisting of sitagliptin, vildagliptin, saxagliptin, linagliptin, gemigliptin, anagliptin, teneligliptin, alogliptin, trelagliptin, dutogliptin, omarigliptin, berberine, lupeol, and any combination thereof. In some embodiments, the sodium-glucose cotransporter-2 (SGLT2) inhibitor or gliflozin is selected from the group consisting of dapagliflozin, empagliflozin, canagliflozin, ipragliflozin, tofogliflozin, ser-20 gliflozin etabonate, remogliflozin etabonate, ertugliflozin, and any combination thereof. In some embodiments, the fibrate is selected from the group consisting of bezafibrate, ciprofibrate, clofibrate, aluminum clofibrate, etofibrate, gemfibrozil, fenofibrate, clinofibrate, and any combination thereof. In some embodiments, the nitric oxide (NO) donor is selected from the group consisting of an 3Q organic nitrate, a diazeniumdiolate (NONOate), an S-nitrosothiol, an active pharmaceutical agent comprising an NO group, an NO-zeolite, arginine, sodium nitroprusside (SNP), and any combination thereof. In some embodiments, the organic nitrate is selected from the group consisting of: glyceryl trinitrate, isosorbide mononitrate, pentaerythritol tetranitrate, and BiDil (isosorbide dinitrate with hydralazine).

In some embodiments of any of the methods disclosed herein, the ActRII polypeptide is administered to the patient every week, every two weeks, every three weeks, or every four weeks. In some embodiments, the ActRII polypeptide is administered to the patient every three weeks. In some embodiments, the polypeptide is administered at a dosing range of 0.1 mg/kg to 2.0 mg/kg.

In some aspects, the disclosure provides a kit comprising a lyophilized polypeptide and an injection device, wherein the polypeptide is an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the kit is used to treat a subject suffering from a disease or condition, wherein the disease or condition is selected from the group consisting of pre-diabetes mellitus, type 1 diabetes mellitus, type 2 diabetes mellitus, complications associated with diabetes mellitus, obesity, IGT, impaired fasting blood glucose (IFG), high glucose levels, glucose intolerance, hyperglycemia, postprandial hyperglycemia, hyperlipidemia, dyslipidemia, insulin resistance, fatty liver, NAFLD, NASH, hepatic steatosis, arteriosclerosis, hypertension, polycystic ovary syndrome, and any combination thereof.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 shows an alignment of extracellular domains of human ActRIIB (SEQ ID NO: 31) and human ActRIIA (SEQ ID NO: 2) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIA proteins and human ActRIIA (SEQ ID NOs: 6-10 and 36-38).

FIG. 3 shows multiple sequence alignment of Fc domains from human IgG isotypes using Clustal 2.1. Hinge regions are indicated by dotted underline. Double underline indicates examples of positions engineered in IgG1 Fc (SEQ ID NO: 32) to promote asymmetric chain pairing and the corresponding positions with respect to other isotypes IgG2 (SEQ ID NO: 33), IgG3 (SEQ ID NO: 34) and IgG4 (SEQ ID NO: 35).

FIGS. 4A and 4B show the purification of ActRIIA-hFc expressed in CHO cells. The protein purifies as a single, well-defined peak as visualized by sizing column (FIG. 4A) and Coomassie stained SDS-PAGE (FIG. 4B) (left lane: molecular weight standards; right lane: ActRIIA-hFc).

FIGS. 5A and 5B show the binding of ActRIIA-hFec to activin (FIG. 5A) and GDF-11 (FIG. 5B), as measured by Biacore^{T}M assay.

FIG. 6 shows components of a kit comprising a lyophilized polypeptide and an injection device. A vial (1) holds lyophilized polypeptide, reconstituted sterile injectable solution, or sterile injectable solution. A prefilled syringe (2) containing a reconstitution solution is used to reconstitute lyophilized polypeptide from (1) into a sterile injectable solution. A vial adapter (3) couples the vial (1) to the pre-filled syringe (2) via attachment to the vial at one end, and attachment to the pre-filled syringe at an opposite end. A syringe (4) and needle (5) are provided for administration of sterile injectable solution. Swab wipes (6) are provided for sterilization of individual kit components.

FIGS. 7-10 show that sotatercept analog RAP-O11 (an ActRIIA-mFc polypeptide) improves diastolic dysfunction in a rat model of left ventricular diastolic dysfunction (also referred to as HEpEF) Group 2 (subgroup 2.2) pulmonary hypertension (PH). The experimental strategy used to test the preventative effects of RAP-O11 in the rat model of HEpEF is shown in FIG. 7. FIGS. 8-10 show endpoints for left ventricular function, including the left ventricular ejection fraction (FIG. 8); LV diastolic function parameters E/E′ [Ratio of mitrial inflow velocity (E) to mitrial annular velocity (E′)] (FIG. 9); and isovolumetric relaxation time (IVRT) (FIG. 10). Statistical significance (p value) is depicted as *p<0.05, **p<0.01, and ***p<0.001.

FIGS. 11-13 show that sotatercept analog RAP-011 (an ActRIIA-mFc polypeptide) reduces left heart remodeling in a rat model of left ventricular diastolic dysfunction (also referred to as HEpEF) Group 2 (subgroup 2.2) pulmonary hypertension (PH). FIGS. 11-13 show endpoints for left heart remodeling, including changes in ratio of heart weight to tibia length (HW/TL) (FIG. 11); interventricular septal dimension at diastole (IVSd) (FIG. 12); and left ventricular mass (LVM) (FIG. 13). Statistical significance (p value) is depicted as *p<0.05, **p<0.01, and ***p<0.001.

FIGS. 14-16 show that sotatercept analog RAP-O11 (an ActRIIA-mFc polypeptide) reduces right ventricular systolic pressure (RVSP) and improves right ventricular function in a rat model of left ventricular diastolic dysfunction (also referred to as HEpEF) Group 2 (subgroup 2.2) pulmonary hypertension (PH). FIGS. 14-16 show endpoints for right ventricular function, including changes in right ventricular free wall thickness (FIG. 14); pulmonary artery acceleration time (PAAT) (FIG. 15); and right ventricular systolic pressure (RVSP) (FIG. 16). Statistical significance (p value) is depicted as *p<0.05 and **p<0.01.

FIGS. 17-19 show that sotatercept analog RAP-011 (an ActRIIA-mFc polypeptide) significantly reduced the fibrosis in LV, RV and lung in a rat model of left ventricular diastolic dysfunction (also referred to as HEpEF) Group 2 (subgroup 2.2) pulmonary hypertension (PH). FIGS. 17-19 show a reduction in fibrosis, including changes in left ventricular fibrosis (FIG. 17); right ventricular fibrosis (FIG. 18); and lung fibrosis (FIG. 19). Statistical significance (p value) is depicted as *p<0.05 and **p<0.01.

FIGS. 20-23 show that sotatercept analog RAP-011 (an ActRIIA-mFc polypeptide) significantly improves hyperglycemia and glucose intolerance in a rat model of left ventricular diastolic dysfunction (also referred to as HEpEF) Group 2 (subgroup 2.2) pulmonary hypertension (PH). FIGS. 20-23 show endpoints for hyperglycemia and glucose intolerance, including changes in body weight (FIG. 20); fasting glucose (FIG. 21); blood glucose (FIG. 22); and glucose/creatine ratio (FIG. 23). Statistical significance (p value) is depicted as *p<0.05, **p<0.01, and ***p<0.001.

FIGS. 24-27 show that sotatercept analog RAP-011 (an ActRIIA-mFc polypeptide) reduces left heart remodeling in a rat model of left ventricular diastolic dysfunction (also referred to as HEpEF) Group 2 (subgroup 2.2) pulmonary hypertension (PH). These figures also show the comparative effects of empagliflozin (Empa) in this model. FIGS. 24-27 show endpoints for left heart remodeling, including changes in ratio of heart weight to tibia length (HW/TL) (FIG. 24); left ventricular mass (LVM) (FIG. 25); LV diastolic function parameters E/E′ [Ratio of mitrial inflow velocity (E) to mitrial annular velocity (E′)] (FIG. 26); and isovolumetric relaxation time (IVRT) (FIG. 27). Statistical significance (p value) is depicted as *p<0.05, **p<0.01, ***p<0.001, and ****p<0.0001.

FIGS. 28-31 show that sotatercept analog RAP-011 (an ActRIIA-mFc polypeptide) reduces right ventricular remodeling and reduces pulmonary hypertension in a rat model of left ventricular diastolic dysfunction (also referred to as HEpEF) Group 2 (subgroup 2.2) pulmonary hypertension (PH). These figures also show the comparative effects of empagliflozin (Empa) in this model. FIGS. 28-31 show endpoints for right ventricular function and hypertension, including changes in right ventricular free wall thickness (RVFWT) (FIG. 28); pulmonary artery acceleration time (PAAT) (FIG. 29); tricuspid annular plane systolic excursion (TAPSE) (FIG. 30); and mean pulmonary arterial pressure (mPAP) (FIG. 31). Statistical significance (p value) is depicted as *p<0.05, **p<0.01, ***p<0.001, and ****p<0.0001.

FIGS. 32-34 show that sotatercept analog RAP-011 (an ActRIIA-mFc polypeptide) significantly reduced the fibrosis in LV, RV and lung in a rat model of left ventricular diastolic dysfunction (also referred to as HEpEF) Group 2 (subgroup 2.2) pulmonary hypertension (PH). These figures also show the comparative effects of empagliflozin (Empa) in this model. FIGS. 32-34 show a reduction in fibrosis, including changes in left ventricular fibrosis (FIG. 32); right ventricular fibrosis (FIG. 33); and lung fibrosis (FIG. 34). Statistical significance (p value) is depicted as *p<0.05, ***p<0.001, and ****p<0.0001.

FIGS. 35-36 show that sotatercept analog RAP-011 (an ActRIIA-mFc polypeptide) significantly improves hyperglycemia and glucose intolerance in a rat model of left ventricular diastolic dysfunction (also referred to as HEpEF) Group 2 (subgroup 2.2) pulmonary hypertension (PH). These figures also show the comparative effects of empagliflozin (Empa) in this model. FIGS. 35-36 show endpoints for hyperglycemia and glucose intolerance, including fasting glucose (FIG. 35); and blood glucose (FIG. 36). Statistical significance (p value) is depicted as *p<0.05 and ****p<0.0001.

### DETAILED DESCRIPTION

### 1. Overview

The present disclosure relates to compositions and methods of treating hyperglycemia and hyperglycemia associated disorders comprising administering to a patient in need thereof an effective amount of an ActRII polypeptide as described herein. In certain embodiments, the present disclosure provides methods of treating or preventing hyperglycemia in an individual in need thereof through administering to the individual a therapeutically effective amount of an ActRII polypeptide as described herein. In certain embodiments, the present disclosure provides methods of treating or preventing hyperglycemia associated with pulmonary hypertension in an individual in need thereof comprising administering to the individual a therapeutically effective amount of an ActRII polypeptide as described herein. In certain embodiments, the present disclosure provides methods of treating or preventing a glucose metabolism disease in an individual in need thereof through administering to the individual a therapeutically effective amount of an ActRII polypeptide as described herein. In some embodiments, the glucose metabolism disease is selected from the group consisting of glucose intolerance, insulin resistance, impaired glucose tolerance (IGT), and impaired fasting glucose.

Hyperglycemia is caused by a dysregulation in glucose homeostasis and results in a subject having higher than normal blood glucose levels. Some of the factors which contribute to hyperglycemia include reduced insulin secretion, decreased glucose utilization, and increased glucose production. Insulin is the most important regulator of glucose homeostasis as it stimulates the utilization of dietary glucose by peripheral tissues while also repressing hepatic glucose production.

Decreased insulin production and/or reduced insulin sensitivity are important contributing factors to the development of hyperglycemia and they represent the underlying abnormalities of diabetes. In addition to decreased insulin secretion, diabetes is also characterized by impaired glucagon production, which can predispose to the risk of hypoglycemia in these patients.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Polypeptides

In certain aspects, the disclosure relates to ActRII polypeptides and uses thereof (e.g., treating, preventing, or reducing the progression rate and/or severity of hyperglycemia or one or more complications of hyperglycemia). As used herein, the term “ActRII” refers to the family of type II activin receptors. This family includes activin receptor type IIA (ActRIIA) and activin receptor type IIB (ActRIIB).

In certain embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in anyone of SEQ ID NOs: 1, 2, 3, 23, 27, 30, and 41. In other embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in anyone of SEQ ID NOs: 31, 39, and 40. As used herein, the term “ActRII” refers to a family of ActRIIA proteins, a family of ActRIIB proteins, or combinations and/or variants thereof. The ActRII polypeptides can be derived from any species and include variants derived from such ActRII proteins by mutagenesis or other modification. Reference to ActRII herein is understood to be a reference to any one of the currently identified forms. Members of the ActRII family are generally transmembrane proteins, composed of a ligand-binding extracellular domain comprising a cysteine-rich region, a transmembrane domain, and a cytoplasmic domain with predicted serine/threonine kinase activity.

The term ActRII polypeptide includes polypeptides comprising any naturally occurring polypeptide of an ActRII family member as well as any variants thereof (including mutants, fragments, fusions, and peptidomimetic forms) that retain a useful activity. Examples of such variant ActRII polypeptides are provided throughout the present disclosure as well as in International Patent Application Publication Nos. WO 2006/012627, WO 2007/062188, WO 2008/097541, WO 2010/151426, and WO 2011/020045, which are incorporated herein by reference in their entirety. Numbering of amino acids for all ActRII-related polypeptides described herein is based on the numbering of the human ActRII precursor protein sequence provided below (SEQ ID NO: 1), unless specifically designated otherwise.

The canonical human ActRII precursor protein sequence is as follows:

| (SEQ ID NO: 1) | |
|---|---|
| 1 | MGAAAKLAFA VFLISCSSGA ILGRSETQEC LFFNANWEKD RT [symbol] QTGVEPC |
| 51 | YGDKDKRRHC FATWK [symbol] ISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GREGCVWKAQ LLNEYVAVKI FPIQDKQSWQ NEYEVYSLPG MKHENILQFI |
| 251 | GAEKRGTSVD VDLWLITAFH EKGSLSDELK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLTNIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed (mature) extracellular human ActRII polypeptide sequence is as follows:

(SEQ ID NO: 2)
ILGRSETQECLFFNANWEKDRINQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPP

The C-terminal “tail” of the extracellular domain is indicated by single underline. The sequence with the “tail” deleted (a Δ15 sequence) is as follows:

(SEQ ID NO: 3)
ILGRSETQECLFFNANWEKDRINQTGVEPCYGDKDKRRHCFATWKNISG
SIEIVKQGCWLDDINCYDRTDCVEKKDSPEVYFCCCEGNMCNEKFSYFP
EM

The nucleic acid sequence encoding human ActRII precursor protein is shown below (SEQ ID NO: 4), see Genbank Reference Sequence NM_001616.4. The signal sequence is underlined.

| (SEQ ID NO: 4) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

The nucleic acid sequence encoding processed soluble (extracellular) human ActRII polypeptide is as follows:

| (SEQ ID NO: 5) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

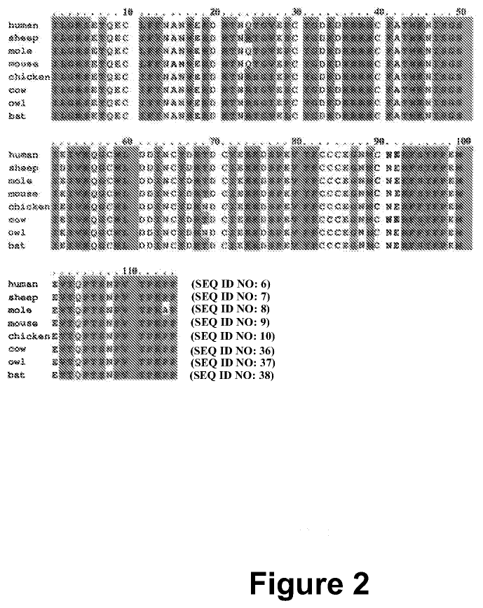

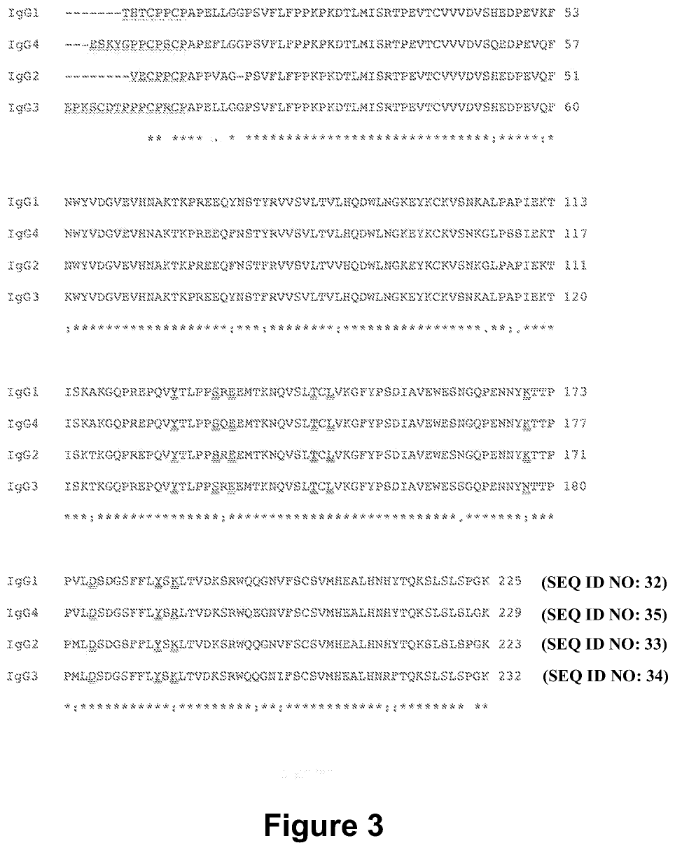

ActRII is well-conserved among vertebrates, with large stretches of the extracellular domain completely conserved. For example, FIG. 2 depicts a multi-sequence alignment of a human ActRII extracellular domain compared to various ActRII orthologs. Many of the ligands that bind to ActRII are also highly conserved. Accordingly, from these alignments, it is possible to predict key amino acid positions within the ligand-binding domain that are important for normal ActRII-ligand binding activities as well as to predict amino acid positions that are likely to be tolerant to substitution without significantly altering normal ActRII-ligand binding activities. Therefore, an active, human ActRII variant polypeptide useful in accordance with the presently disclosed methods may include one or more amino acids at corresponding positions from the sequence of another vertebrate ActRII, or may include a residue that is similar to that in the human or other vertebrate sequences.

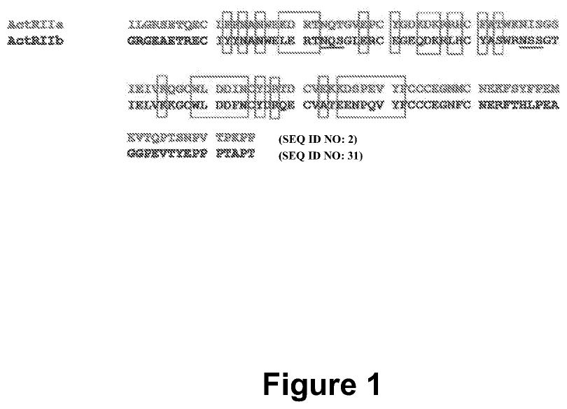

An alignment of the amino acid sequences of human ActRIIA extracellular domain and human ActRIIB extracellular domain is illustrated in FIG. 1. This alignment indicates amino acid residues within both receptors that are believed to directly contact ActRII ligands. For example, the composite ActRII structures indicated that the ActRIIA-ligand binding pocket is defined, in part, by residues F31, N33, N35, K38 through T41, E47, Y50, K53 through K55, R57, H58, F60, T62, K74, W78 through N83, Y85, R87, E92, and K94 through F101. At these positions, it is expected that conservative mutations will be tolerated.

Without meaning to be limiting, the following examples illustrate this approach to defining an active ActRII variant. As illustrated in FIG. 2, F13 in the human extracellular domain is Y in Ovis aries (SEQ ID NO: 7), Gallus gallus (SEQ ID NO: 10), Bos Taurus (SEQ ID NO: 36), Tyto alba (SEQ ID NO: 37), and Myotis davidii (SEQ ID NO: 38) ActRII, indicating that aromatic residues are tolerated at this position, including F, W, and Y. Q24 in the human extracellular domain is R in Bos Taurus ActRII, indicating that charged residues will be tolerated at this position, including D, R, K, H, and E. S95 in the human extracellular domain is F in Gallus gallus and Tyto alba ActRII, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRII, indicating that acidic residues are tolerated at this position, including D and E. P29 in the human extracellular domain is relatively poorly conserved, appearing as S in Ovis aries ActRII and L in Myotis davidii ActRII, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1): 97-108; Greenwald et al. (1999) Nature Structural Biology 6(1): 18-22; Allendorph et al. (2006) PNAS 103(20): 7643-7648; Thompson et al. (2003) The EMBO Journal 22(7): 1555-1566; as well as U.S. Pat. Nos. 7,709,605, 7,612,041, and 7,842,663]. For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. In addition to the teachings herein, these references provide ample guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRII, as demarcated by the outermost of these conserved cysteines, corresponds to positions 30-110 of SEQ ID NO: 1 (ActRII precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRII extracellular domains truncations include SEQ ID NOs: 2 and 3.

Accordingly, a general formula for an active portion (e.g., ligand binding) of ActRII is a polypeptide that comprises, consists essentially of, or consists of amino acids 30-110 of SEQ ID NO: 1. Therefore ActRII polypeptides may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 1. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 1, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1. Thus, in some embodiments, an ActRII polypeptide may comprise, consist essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1. Optionally, ActRII polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1, and comprises no more than 1, 2, 5, 10 or 15 conservative amino acid changes in the ligand-binding pocket.

In certain embodiments, the disclosure relates to an ActRII polypeptide, which includes fragments, functional variants, and modified forms thereof as well as uses thereof (e.g., treating, preventing, or reducing hyperglycemia). Preferably, ActRII polypeptides are soluble (e.g., an extracellular domain of ActRII). In some embodiments, ActRII polypeptides inhibit (e.g., Smad signaling) of one or more GDF/BMP ligands (e.g., GDF11, GDF8, activin A, activin B, GDF3, BMP4, BMP6, BMP10, and/or BMP15). In some embodiments, ActRII polypeptides bind to one or more GDF/BMP ligands (e.g., GDF11, GDF8, activin A, activin B, GDF3, BMP4, BMP6, BMP10, and/or BMP15). In some embodiments, ActRII polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 1. In certain embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 23, 27, 30, and 41.

In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some alternative embodiments, the ActRII polypeptide (e.g., SEQ ID NO: 23) lacks the C-terminal lysine. In some embodiments, the ActRII polypeptide lacking the C-terminal lysine is SEQ ID NO: 41. In some embodiments, the ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered a combination of SEQ ID NO: 23 and SEQ ID NO: 41.

In certain aspects, the present disclosure relates to ActRII polypeptides. In some embodiments, ActRII traps of the present disclosure are variant ActRII polypeptides (e.g., ActRIIA polypeptides, ActRIIB polypeptides, or combinations thereof) that comprise one or more mutations (e.g., amino acid additions, deletions, substitutions, and combinations thereof) in the extracellular domain (also referred to as the ligand-binding domain) of an ActRII polypeptide (e.g., a “wild-type” or unmodified ActRII polypeptide) such that the variant ActRII polypeptide has one or more altered ligand-binding activities than the corresponding wild-type ActRII polypeptide. In preferred embodiments, variant ActRII polypeptides of the present disclosure retain at least one similar activity as a corresponding wild-type ActRII polypeptide. For example, preferable ActRII polypeptides bind to and inhibit (e.g. antagonize) the function of GDF11 and/or GDF8. In some embodiments, ActRII polypeptides of the present disclosure further bind to and inhibit one or more of ligand of GDF/BMP (e.g., GDF11, GDF8, activin A, activin B, GDF3, BMP4, BMP6, BMP10, and/or BMP15). Accordingly, the present disclosure provides ActRII polypeptides that have an altered binding specificity for one or more ActRII ligands.

To illustrate, one or more mutations may be selected that increase the selectivity of the altered ligand-binding domain for GDF11 and/or GDF8 over one or more ActRII-binding ligands such as activins (activin A or activin B), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

In certain embodiments, the present disclosure contemplates specific mutations of an ActRII polypeptide so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ActRII polypeptide as well as truncation mutants. Pools of combinatorial mutants are especially useful for identifying functionally active (e.g., GDF/BMP ligand binding) ActRII sequences. The purpose of screening such combinatorial libraries may be to generate, for example, polypeptides variants, which have altered properties, such as altered pharmacokinetic or altered ligand binding. A variety of screening assays are provided below, and such assays may be used to evaluate variants. For example, ActRII variants may be screened for ability to bind to one or more GDF/BMP ligands (e.g., GDF11, GDF8, activin A, activin B, GDF3, BMP4, BMP6, BMP10, and/or BMP15), to prevent binding of a GDF/BMP ligand to an ActRII polypeptide, as well as heteromultimers thereof, and/or to interfere with signaling caused by an GDF/BMP ligand.

The activity of ActRII polypeptides or variants thereof may also be tested in a cell-based or in vivo assay. For example, the effect of an ActRII polypeptide on the expression of genes involved in PH pathogenesis may be assessed. This may, as needed, be performed in the presence of one or more recombinant ligand proteins (e.g., GDF11, GDF8, activin A, activin B, GDF3, BMP4, BMP6, BMP10, and/or BMP15), and cells may be transfected so as to produce an ActRII polypeptide, and optionally, an GDF/BMP ligand. Likewise, an ActRII polypeptide may be administered to a mouse or other animal and effects on PH pathogenesis may be assessed using art-recognized methods. Similarly, the activity of an ActRII polypeptide or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference ActRII polypeptide. Such variants, when expressed from recombinant DNA constructs, can be used in gene therapy protocols. Likewise, mutagenesis can give rise to variants which have intracellular half-lives dramatically different than the corresponding unmodified ActRII polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the ActRII polypeptide.

A combinatorial library may be produced by way of a degenerate library of genes encoding a library of polypeptides which each include at least a portion of potential ActRII polypeptide sequences. For instance, a mixture of synthetic oligonucleotides can be enzymatically ligated into gene sequences such that the degenerate set of potential ActRII encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815].

Alternatively, other forms of mutagenesis can be utilized to generate a combinatorial library. For example, ActRII polypeptides of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of ActRII polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRII polypeptides. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include ligand (e.g., GDF11, GDF8, activin A, activin B, GDF3, BMP4, BMP6, BMP10, and/or BMP15) binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of ActRII polypeptides that can be used as guidance for generating and using other variant ActRII polypeptides within the scope of the disclosure provided herein.

In certain embodiments, functionally active fragments of ActRII polypeptides of the present disclosure can be obtained by screening polypeptides recombinantly produced from the corresponding fragment of the nucleic acid encoding an ActRII polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII receptors and/or one or more ligands (e.g., GDF11, GDF8, activin A, activin B, GDF3, BMP4, BMP6, BMP10, and/or BMP15).

In certain embodiments, ActRII polypeptides of the present disclosure may further comprise post-translational modifications in addition to any that are naturally present in the ActRII polypeptide. Such modifications include, but are not limited to, acetylation, carboxylation, glycosylation, phosphorylation, lipidation, and acylation. As a result, the ActRII polypeptide may contain non-amino acid elements, such as polyethylene glycols, lipids, polysaccharide or monosaccharide, and phosphates. Effects of such non-amino acid elements on the functionality of a ligand trap polypeptide may be tested as described herein for other ActRII variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, W138, NIH-3T3 or HEK293) have specific cellular machinery and characteristic mechanisms for such post-translational activities and may be chosen to ensure the correct modification and processing of the ActRII polypeptides.

In certain aspects, ActRII polypeptides of the present disclosure include fusion proteins having at least a portion (domain) of an ActRII polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), and human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ActRII polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (i.e., domains that confer an additional biological function) including, for example constant domains from immunoglobulins (e.g., Fc domains).

In certain aspects, ActRII polypeptides of the present disclosure contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising an ActRII polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain preferred embodiments, an ActRII polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., a Fc domain) are known to confer desirable pharmacokinetic properties on a wide range of proteins. Likewise, fusions to human serum albumin can confer desirable properties.

An example of a native amino acid sequence that may be used for the Fc portion of human IgG1 (G1Fc) is shown below (SEQ ID NO: 11). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 11. Naturally occurring variants in G1Fc would include E134D and M136L substitutions according to the numbering system used in SEQ ID NO: 11 (see Uniprot P01857).

| (SEQ ID NO: 11) | |
|---|---|
| 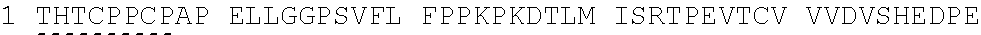 | |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PREEMTKNQ VSLTCLVRGF |
| 151 | YPSDIAVEWE SNGQPENNYK TTPPVLDSDG SFFLYSKLTV DKSRWQQGNV |
| 201 | GFSCSVMHEAL HNHYTQKSLS LSPGK |

Optionally, the IgG1 Fe domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 12). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 12.

| (SEQ ID NO: 12) | |
|---|---|
| 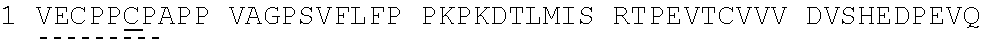 | |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 13) contains a short hinge region consisting of a single 15-residue segment, whereas the second G3Fc sequence (SEQ ID NO: 14) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 13 and 14.

| (SEQ ID NO: 14) | |
|---|---|
| 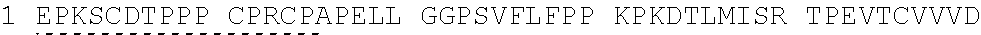 | |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| 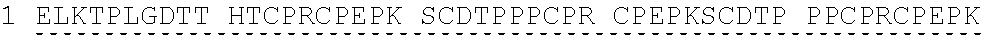 | |
| 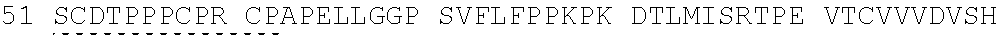 | |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include one or more of E68Q, P76L, E79Q, Y81F, D97N, N100D, T124A, S169N, S169del, F221Y substitutions when converted to the numbering system used in SEQ ID NO: 13, and the present disclosure provides fusion proteins comprising G3Fc domains containing one or more of these variations. In addition, the human immunoglobulin IgG3 gene (IGHG3) shows a structural polymorphism characterized by different hinge lengths (see Uniprot P01859). Specifically, variant WIS is lacking most of the V region and all of the CHi region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 15). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15.

| (SEQ ID NO: 15) | |
|---|---|
| 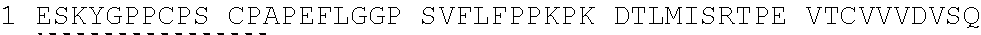 | |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

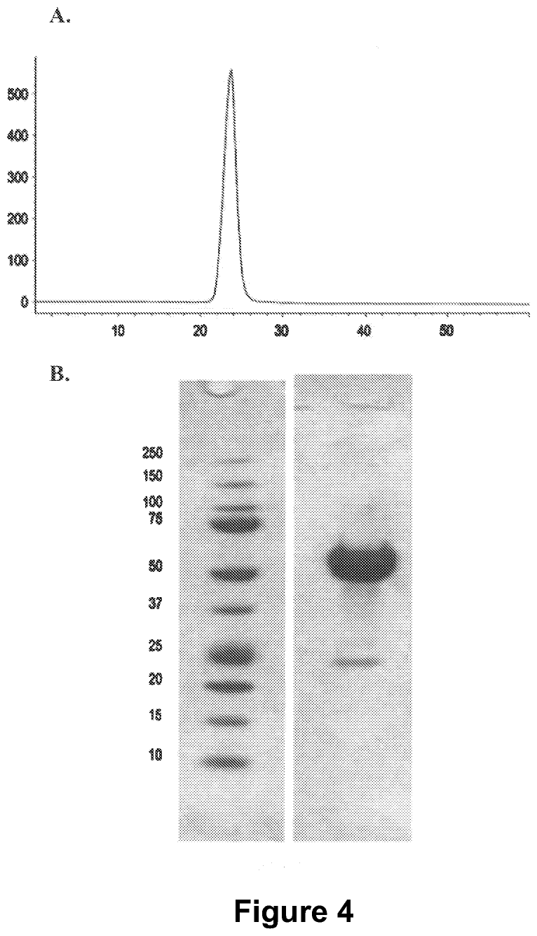

A variety of engineered mutations in the Fe domain are presented herein with respect to the G1Fc sequence (SEQ ID NO: 11), and analogous mutations in G2Fc, G3Fc, and G4Fc can be derived from their alignment with G1Fc in FIG. 4. Due to unequal hinge lengths, analogous Fc positions based on isotype alignment (FIG. 4) possess different amino acid numbers in SEQ ID NOs: 11, 12, 13, 14, and 15. It can also be appreciated that a given amino acid position in an immunoglobulin sequence consisting of hinge, C_{H}2, and C_{H}3 regions (e.g., SEQ ID NOs: 11, 12, 13, 14, and 15) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 11), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | |
|---|---|---|
| (Numbering begins at | constant domain | IgG1 heavy chain |
| first threonine in | (Numbering begins at | (EU numbering scheme |
| hinge region) | C_{H}1) | of Kabat et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

Various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing [Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605].

It is understood that different elements of the fusion proteins (e.g., immunoglobulin Fc fusion proteins) may be arranged in any manner that is consistent with desired functionality. For example, an ActRII polypeptide domain may be placed C-terminal to a heterologous domain, or alternatively, a heterologous domain may be placed C-terminal to an ActRII polypeptide domain. The ActRII polypeptide domain and the heterologous domain need not be adjacent in a fusion protein, and additional domains or amino acid sequences may be included C- or N-terminal to either domain or between the domains.

For example, an ActRII receptor fusion protein may comprise an amino acid sequence as set forth in the formula A-B-C. The B portion corresponds to an ActRII polypeptide domain. The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 16), GGGG (SEQ ID NO: 17), TGGGG (SEQ ID NO: 18), SGGGG (SEQ ID NO: 19), TGGG (SEQ ID NO: 20), SGGG (SEQ ID NO: 21), or GGGGS (SEQ ID NO: 22) singlets, or repeats. In certain embodiments, an ActRII fusion protein comprises an amino acid sequence as set forth in the formula A-B-C, wherein A is a leader (signal) sequence, B consists of an ActRII polypeptide domain, and C is a polypeptide portion that enhances one or more of in vivo stability, in vivo half-life, uptake/administration, tissue localization or distribution, formation of protein complexes, and/or purification. In certain embodiments, an ActRII fusion protein comprises an amino acid sequence as set forth in the formula A-B-C, wherein A is a TPA leader sequence, B consists of an ActRII receptor polypeptide domain, and C is an immunoglobulin Fc domain. Preferred fusion proteins comprise the amino acid sequence set forth in any one of SEQ ID NOs: 23, 27, 30, and 41.

In preferred embodiments, ActRII polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides to be used in accordance with the methods described herein are recombinant polypeptides.

ActRII polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRII polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ActRII polypeptides using chemical cleavage (e.g., cyanogen bromide, hydroxylamine, etc.).

### 3. Nucleic Acids Encoding ActRII Polypeptides

In certain embodiments, the present disclosure provides isolated and/or recombinant nucleic acids encoding ActRII polypeptides (including fragments, functional variants, and fusion proteins thereof).

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII polypeptides of the disclosure are understood to include nucleic acids that are variants of any one of SEQ ID NOs: 4, 5, or 28. Variant nucleotide sequences include sequences that differ by one or more nucleotide substitutions, additions, or deletions including allelic variants, and therefore, will include coding sequence that differ from the nucleotide sequence designated in any one of SEQ ID NOs: 4, 5, or 28.

In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to any one of SEQ ID NOs: 4, 5, or 28. One of ordinary skill in the art will appreciate that nucleic acid sequences that are at least 70%, 75%, 80%, 85%, 90%, 91%, 92%, 93%, 94% 95%, 96%, 97%, 98%, 99%, or 100% identical to the sequences complementary to SEQ ID NOs: 4, 5, or 28, and variants thereof, are also within the scope of the present disclosure. In further embodiments, the nucleic acid sequences of the disclosure can be isolated, recombinant, and/or fused with a heterologous nucleotide sequence, or in a DNA library.

In other embodiments, nucleic acids of the present disclosure also include nucleotide sequences that hybridize under highly stringent conditions to the nucleotide sequence designated in SEQ ID NOs: 4, 5, or 28, complement sequences of SEQ ID NOs: 4, 5, or 28, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids that differ from the nucleic acids as set forth in SEQ ID NOs: 4, 5, or 28 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art and can be used in a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and can vary with the host cell used.

In certain aspects, the subject nucleic acid disclosed herein is provided in an expression vector comprising a nucleotide sequence encoding an ActRII polypeptide and operably linked to at least one regulatory sequence. Regulatory sequences are art-recognized and are selected to direct expression of the ActRII polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding an ActRII polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In a preferred embodiment, a vector will be designed for production of the subject ActRII polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ActRII polypeptides in cells propagated in culture, e.g., to produce proteins, including fusion proteins or variant proteins, for purification.

This disclosure also pertains to a host cell transfected with a recombinant gene including a coding sequence for one or more of the subject ActRII polypeptides. The host cell may be any prokaryotic or eukaryotic cell. For example, an ActRII polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRII polypeptides. For example, a host cell transfected with an expression vector encoding an ActRII polypeptide can be cultured under appropriate conditions to allow expression of the ActRII polypeptide to occur. The polypeptide may be secreted and isolated from a mixture of cells and medium containing the polypeptide. Alternatively, the ActRII polypeptide may be retained cytoplasmically or in a membrane fraction and the cells harvested, lysed and the protein isolated. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRII polypeptides, and affinity purification with an agent that binds to a domain fused to the ActRII polypeptide (e.g., a protein A column may be used to purify an ActRII-Fc fusion proteins). In some embodiments, the ActRII polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII polypeptide, can allow purification of the expressed fusion protein by affinity chromatography using a Ni²⁺ metal resin. The purification leader sequence can then be subsequently removed by treatment with enterokinase to provide the purified ActRII polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### Methods of Use

In part, the present disclosure relates to methods of treating hyperglycemia and complications of hyperglycemia (e.g., diabetes mellitus) comprising administering to a patient in need thereof an effective amount of an ActRII polypeptide as described herein. In some embodiments, the present disclosure relates to methods of treating, preventing, or reducing the progression rate and/or severity of one or more complications of hyperglycemia comprising administering to a patient in need thereof an effective amount of an ActRII polypeptide as described herein. In some embodiments, the hyperglycemia is associated with pulmonary hypertension (PH). In some embodiments, the ActRII polypeptide is administered at a dosing range of 0.1 mg/kg to 2.0 mg/kg. In some embodiments, the one or more complications of hyperglycemia is selected from the group consisting of cataracts, microvascular disease, macrovascular disease, nephropathy, diabetic nephropathy, glomerulosclerosis, retinopathy, diabetic retinopathy, choroidal neovascularisation, coronary artery disease, cerebrovascular disease, peripheral vascular disease, NAFL disease, NASH, neuropathy, diabetic neuropathy, diabetic pain, tissue ischaemia, diabetic foot, diabetic ulcer, and arteriosclerosis. These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., hyperglycemia). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample. In some embodiments, the disclosure relates to methods of administering an ActRII polypeptide to a patient in need of treatment (e.g., a “patient in need thereof”). Such patients in need of treatment with an ActRII polypeptide patients having a disorder or condition disclosed in the instant application including, but not limited to, hyperglycemia, a complication of hyperglycemia, insulin sensitivity, insulin resistance, decreased glucose utilization, glucose metabolism disease, fibrosis, a fibrosis associated disorder, diabetes, or a diabetes associated disorder.

In general, treatment or prevention of a disease or condition as described in the present disclosure (e.g., hyperglycemia) is achieved by administering one or more ActRII polypeptides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of an ActRII polypeptide, in combination with one or more additional active agents or other supportive therapy for treating or preventing a disease or condition (e.g., hyperglycemia). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more ActRII polypeptides of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII polypeptide of the present disclosure with the additional active agent or therapy and/or the desired effect.

The embodiments described herein provide compositions and methods for treating and preventing disorders of hyperglycemia, insulin resistance, glucose intolerance, diabetes, fibrosis, hyperlipidemia, dyslipidemia, and their associated conditions.

### Hyperglycemia and Insulin Resistance

Hyperglycemia is caused by a dysregulation in glucose homeostasis and results in a subject having higher than normal blood glucose levels. Some of the factors which contribute to hyperglycemia include reduced insulin secretion, decreased glucose utilization, and increased glucose production. Insulin is the most important regulator of glucose homeostasis as it stimulates the utilization of dietary glucose by peripheral tissues while also repressing hepatic glucose production.

Decreased insulin production and/or reduced insulin sensitivity are important contributing factors to the development of hyperglycemia and they represent the underlying abnormalities of diabetes. In addition to decreased insulin secretion, diabetes is also characterized by impaired glucagon production, which can predispose to the risk of hypoglycemia in these patients.

In certain aspects, the disclosure relates to methods of treating, preventing, or reducing the progression rate and/or severity of hyperglycemia associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). As can be appreciated, ActRII polypeptides include SEQ ID NO: 32 and other variants described herein. In some embodiments, the disclosure relates to methods of treating, preventing, or reducing the progression rate and/or severity of hyperglycemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the disclosure relates to methods of treating, preventing, or reducing the progression rate and/or severity of hyperglycemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of hyperglycemia associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of hyperglycemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the one or more complications of hyperglycemia is selected from the group consisting of pre-diabetes mellitus, type 1 diabetes mellitus, type 2 diabetes mellitus, complications associated with diabetes mellitus, cataracts, microvascular disease, macrovascular disease, nephropathy, diabetic nephropathy, glomerulosclerosis, retinopathy, diabetic retinopathy, choroidal neovascularisation, coronary artery disease, cerebrovascular disease, peripheral vascular disease, NAFLD, NASH, neuropathy, diabetic neuropathy, diabetic pain, tissue ischaemia, diabetic foot, diabetic ulcer, arteriosclerosis, hepatic steatosis, hypertension, and polycystic ovary syndrome. In some embodiments, the disclosure relates to methods of treating, preventing, or reducing the progression rate and/or severity of one or more complications of hyperglycemia and insulin resistance or low insulin concentrations, comprising administering to a patient in need thereof an effective amount of an ActRII polypeptide (e.g., an amino acid sequence that is at least 90% identical to an amino acid sequence corresponding to residues 30-110 of SEQ ID NO: 1). While a cluster of signs and symptoms associated may coexist in an individual patient, it many cases only one symptom may dominate, due to individual differences in vulnerability of the many physiological systems affected by insulin resistance. In some embodiments, the patient further has pulmonary hypertension. In some embodiments, the pulmonary hypertension is pulmonary arterial hypertension. In some embodiments, the patient further has decreased renal function. In some embodiments, the method further improves renal function.

In some embodiments, the subject has hyperglycemia associated with destruction of the pancreas. In some embodiments, the destruction of the pancreas is due to chronic pancreatitis, hemochromatosis, pancreatic cancer, or cystic fibrosis. In some embodiments, the subject has hyperglycemia associated with an endocrine disorder. In some embodiments, the endocrine disorder is selected from the group consisting of Cushing syndrome, acromegaly, and pheochromocytoma. In some embodiments, the subject has hyperglycemia associated with the use of medications selected from the group consisting of glucocorticoids, phenytoin, and estrogens. In some embodiments, the subject has hyperglycemia associated with gestational diabetes. In some embodiments, the subject has hyperglycemia associated with total parental nutrition and/or dextrose infusion. In some embodiments, the subject has hyperglycemia following an operative procedure.

In certain aspects, the disclosure relates to methods of treating, preventing, or reducing the progression rate and/or severity of insulin resistance associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). As can be appreciated, ActRII polypeptides include SEQ ID NO: 32 and other variants described herein. In some embodiments, the disclosure relates to methods of treating, preventing, or reducing the progression rate and/or severity of insulin resistance, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of insulin resistance associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the disclosure relates to methods of increasing insulin sensitivity in a patient with pulmonary hypertension, comprising administering to a patient in need thereof an effective amount of an ActRII polypeptide (e.g., an amino acid sequence that is at least 90% identical to an amino acid sequence corresponding to residues 30-110 of SEQ ID NO: 1). As can be appreciated, ActRII polypeptides include SEQ ID NO: 32 and other variants described herein. In some embodiments, the disclosure relates to methods of increasing insulin sensitivity, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the insulin resistance is not insulin resistance induced by trauma. In some embodiments, the patient further has pulmonary hypertension. In some embodiments, the pulmonary hypertension is pulmonary arterial hypertension. In some embodiments, the patient further has decreased renal function. In some embodiments, the method further improves renal function.

In some embodiments, the patient has hyperglycemia and type 2 diabetes. In some embodiments, the patient has hyperglycemia and a family history of type 2 diabetes. In some embodiments, the patient is at risk of developing hyperglycemia (e.g., a family history of type 2 diabetes). In some embodiments, the patient has hyperglycemia and hyperlipidemia. In some embodiments, the patient is at risk of developing hyperglycemia (e.g., patient having hyperlipidemia). In some embodiments, the patient has hyperglycemia and hypertension. In some embodiments, the patient is at risk of developing hyperglycemia (e.g., patient having hypertension). In some embodiments, the patient has hyperglycemia and a history of gestational diabetes. In some embodiments, the patient is at risk of developing hyperglycemia (e.g., a history of gestational diabetes). In some embodiments, the patient has hyperglycemia and polycystic ovarian syndrome. In some embodiments, the patient is at risk of developing hyperglycemia (e.g., patient having polycystic ovarian syndrome). In some embodiments, the patient has hyperglycemia and type 1 diabetes. In some embodiments, the patient has postprandial hyperglycemia.

In certain aspects, the disclosure relates to methods of increasing glucose utilization, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of increasing glucose utilization, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the patient further has pulmonary hypertension. In some embodiments, the pulmonary hypertension is pulmonary arterial hypertension. In some embodiments, the subject further has a condition selected from the group consisting of prediabetes, diabetes mellitus, type 1 diabetes mellitus, and type 2 diabetes mellitus.

The diagnosis of hyperglycemia can be determined based on blood glucose levels using various assessments. For instance, blood glucose greater than 125 mg/dL while fasting and greater than 180 mg/dL 2 hours postprandial is indicative of hyperglycemia. Additionally, a patient with a fasting plasma glucose of 100 mg/dL to 125 mg/dL has impaired glucose tolerance, or pre-diabetes. A patient is termed diabetic with a fasting blood glucose of greater than 125 mg/dL. Patients with normal plasma glucose concentrations have average values of around 90 mg/dl throughout a 24-hour period, post-meal concentration below 140 mg/dl, and minimal values, such as those after moderate fasting or exercise, above 55 mg/dl.

In certain aspects, the disclosure relates to methods of treating hyperglycemia, comprising administering to a patient in need thereof an effective amount of an ActRII polypeptide (e.g., an amino acid sequence that is at least 90% identical to an amino acid sequence corresponding to residues 30-110 of SEQ ID NO: 1), thereby decreasing fasting glucose levels. In some embodiments, the disclosure relates to methods of adjusting one or more parameters in the patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's fasting glucose levels. In some embodiments, the method relates to decreasing the patient's fasting glucose levels by at least 10% (e.g., 10, 15, 20, 25, 30, 35, 40, 45, 50, 55, 60, 65, 70, 75, 80, 85, 90, 95, or 100%). In some embodiments, the method relates to decreasing the patient's fasting glucose levels by at least 10%. In some embodiments, the method relates to decreasing the patient's fasting glucose levels by at least 15%. In some embodiments, the method relates to decreasing the patient's fasting glucose levels by at least 20%. In some embodiments, the method relates to decreasing the patient's fasting glucose levels by at least 25%. In some embodiments, the method relates to decreasing the patient's fasting glucose levels by at least 30%. In some embodiments, the method relates to decreasing the patient's fasting glucose levels by at least 35%. In some embodiments, the method relates to decreasing the patient's fasting glucose levels by at least 40%. In some embodiments, the method relates to decreasing the patient's fasting glucose levels by at least 45%. In some embodiments, the method relates to decreasing the patient's fasting glucose levels by at least 50%. In some embodiments, the method relates to decreasing the patient's fasting glucose levels by at least 55%. In some embodiments, the method relates to decreasing the patient's fasting glucose levels by at least 60%. In some embodiments, the method relates to decreasing the patient's fasting glucose levels by at least 65%. In some embodiments, the method relates to decreasing the patient's fasting glucose levels by at least 70%. In some embodiments, the method relates to decreasing the patient's fasting glucose levels by at least 75%. In some embodiments, the method relates to decreasing the patient's fasting glucose levels by at least 80%. In some embodiments, the method relates to decreasing the patient's fasting glucose levels by at least 85%. In some embodiments, the method relates to decreasing the patient's fasting glucose levels by at least 90%. In some embodiments, the method relates to decreasing the patient's fasting glucose levels by at least 95%. In some embodiments, the method relates to decreasing the patient's fasting glucose levels by 100%. In some embodiments, the method relates to decreasing the patient's fasting glucose levels to less than 125 mg/dL. In some embodiments, the method relates to decreasing the patient's fasting glucose levels to less than 120 mg/dL. In some embodiments, the method relates to decreasing the patient's fasting glucose levels to less than 115 mg/dL. In some embodiments, the method relates to decreasing the patient's fasting glucose levels to less than 110 mg/dL. In some embodiments, the method relates to decreasing the patient's fasting glucose levels to less than 105 mg/dL. In some embodiments, the method relates to decreasing the patient's fasting glucose levels to less than 100 mg/dL. In some embodiments, the method relates to decreasing the patient's fasting glucose levels to less than 95 mg/dL.

In certain aspects, the disclosure relates to methods of treating hyperglycemia, comprising administering to a patient in need thereof an effective amount of an ActRII polypeptide (e.g., an amino acid sequence that is at least 90% identical to an amino acid sequence corresponding to residues 30-110 of SEQ ID NO: 1), wherein blood glucose levels in the patient are reduced two hours after eating. In some embodiments, the method relates to decreasing the patient's blood glucose levels two hours after eating. In some embodiments, the method relates to decreasing the patient's blood glucose levels two hours after eating, wherein the patient's blood glucose levels are decreased by at least 10% (e.g., 10, 15, 20, 25, 30, 35, 40, 45, 50, 55, 60, 65, 70, 75, 80, 85, 90, 95, or 100%) two hours after eating. In some embodiments, the patient's blood glucose levels are decreased two hours after eating by at least 10%. In some embodiments, the patient's blood glucose levels are decreased two hours after eating by at least 15%. In some embodiments, the patient's blood glucose levels are decreased two hours after eating by at least 20%. In some embodiments, the patient's blood glucose levels are decreased two hours after eating by at least 25%. In some embodiments, the patient's blood glucose levels are decreased two hours after eating by at least 30%. In some embodiments, the patient's blood glucose levels are decreased two hours after eating by at least 35%. In some embodiments, the patient's blood glucose levels are decreased two hours after eating by at least 40%. In some embodiments, the patient's blood glucose levels are decreased two hours after eating by at least 45%. In some embodiments, the patient's blood glucose levels are decreased two hours after eating by at least 50%. In some embodiments, the patient's blood glucose levels are decreased two hours after eating by at least 55%. In some embodiments, the patient's blood glucose levels are decreased two hours after eating by at least 60%. In some embodiments, the patient's blood glucose levels are decreased two hours after eating by at least 65%. In some embodiments, the blood glucose levels are decreased two hours after eating by at least 70%. In some embodiments, the patient's blood glucose levels are decreased two hours after eating by at least 75%. In some embodiments, the patient's blood glucose levels are decreased two hours after eating by at least 80%. In some embodiments, the patient's blood glucose levels are decreased two hours after eating by at least 85%. In some embodiments, the patient's blood glucose levels are decreased two hours after eating by at least 90%. In some embodiments, the patient's blood glucose levels are decreased two hours after eating by at least 95%. In some embodiments, the patient's blood glucose levels are decreased two hours after eating by 100%.

In some embodiments, the method relates to decreasing blood glucose levels two hours after eating comprising administering to a patient in need thereof an effective amount of an ActRII polypeptide (e.g., an amino acid sequence that is at least 90% identical to an amino acid sequence corresponding to residues 30-110 of SEQ ID NO: 1), wherein blood glucose levels in the patient are reduced two hours after eating by at least 10 mg/dL (e.g., 10, 20, 30, 40, 50, 60, 70, 80, 90, 100, 110, 120, or 130 mg/dL). In some embodiments, the patient's blood glucose levels are decreased two hours after eating by at least 10 mg/dL. In some embodiments, the patient's blood glucose levels are decreased two hours after eating by at least 20 mg/dL. In some embodiments, the patient's blood glucose levels are decreased two hours after eating by at least 30 mg/dL. In some embodiments, the patient's blood glucose levels are decreased two hours after eating by at least 40 mg/dL. In some embodiments, the patient's blood glucose levels are decreased two hours after eating by at least 50 mg/dL. In some embodiments, the patient's blood glucose levels are decreased two hours after eating by at least 60 mg/dL. In some embodiments, the patient's blood glucose levels are decreased two hours after eating by at least 70 mg/dL. In some embodiments, the patient's blood glucose levels are decreased two hours after eating by at least 80 mg/dL. In some embodiments, the patient's blood glucose levels are decreased two hours after eating by at least 90 mg/dL. In some embodiments, the patient's blood glucose levels are decreased two hours after eating by at least 100 mg/dL. In some embodiments, the patient's blood glucose levels are decreased two hours after eating by at least 110 mg/dL. In some embodiments, the patient's blood glucose levels are decreased two hours after eating by at least 120 mg/dL. In some embodiments, the patient's blood glucose levels are decreased two hours after eating by at least 130 mg/dL.

In certain aspects, the disclosure relates to methods of treating hyperglycemia, comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the glucose/creatine ratio in the patient's urine is decreased after the administration. In some embodiments, the method relates to decreasing the patient's glucose/creatinine ratio. In some embodiments, the glucose/creatinine ratio is measured in the patient's urine. In some embodiments, the method relates to decreasing the patient's glucose/creatinine ratio after administration of the ActRII polypeptide, wherein the patient's glucose/creatinine ratio is decreased to less than 15 mg/mg (e.g., 15, 14, 13, 12, 11, 10, 9, 8, 7, 6, 5, 4, 3, 2, 1 mg/mg). In some embodiments, the patient's glucose/creatinine ratio is decreased to less than 14 mg/mg. In some embodiments, the patient's glucose/creatinine ratio is decreased to less than 13 mg/mg. In some embodiments, the patient's glucose/creatinine ratio is decreased to less than 12 mg/mg. In some embodiments, the patient's glucose/creatinine ratio is decreased to less than 11 mg/mg. In some embodiments, the patient's glucose/creatinine ratio is decreased to less than 10 mg/mg. In some embodiments, the patient's glucose/creatinine ratio is decreased to less than 9 mg/mg. In some embodiments, the patient's glucose/creatinine ratio is decreased to less than 8 mg/mg. In some embodiments, the patient's glucose/creatinine ratio is decreased to less than 7 mg/mg. In some embodiments, the patient's glucose/creatinine ratio is decreased to less than 6 mg/mg. In some embodiments, the patient's glucose/creatinine ratio is decreased to less than 5 mg/mg. In some embodiments, the patient's glucose/creatinine ratio is decreased to less than 4 mg/mg. In some embodiments, the patient's glucose/creatinine ratio is decreased to less than 3 mg/mg. In some embodiments, the patient's glucose/creatinine ratio is decreased to less than 2 mg/mg. In some embodiments, the patient's glucose/creatinine ratio is decreased to less than 1 mg/mg.

In some embodiments, the disclosure relates to methods of decreasing the patient's glucose/creatinine ratio, comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the glucose/creatinine ratio in the patient is decreased by at least 10% (e.g., 10, 15, 20, 25, 30, 35, 40, 45, 50, 55, 60, 65, 70, 75, 80, 85, 90, 95, or 100%). In some embodiments, the patient's glucose/creatinine ratio is decreased by at least 15%. In some embodiments, the patient's glucose/creatinine ratio is decreased by at least 20%. In some embodiments, the patient's glucose/creatinine ratio is decreased by at least 25%. In some embodiments, the patient's glucose/creatinine ratio is decreased by at least 30%. In some embodiments, the patient's glucose/creatinine ratio is decreased by at least 35%. In some embodiments, the patient's glucose/creatinine ratio is decreased by at least 40%. In some embodiments, the patient's glucose/creatinine ratio is decreased by at least 45%. In some embodiments, the patient's glucose/creatinine ratio is decreased by at least 50%. In some embodiments, the patient's glucose/creatinine ratio is decreased by at least 55%. In some embodiments, the patient's glucose/creatinine ratio is decreased by at least 60%. In some embodiments, the patient's glucose/creatinine ratio is decreased by at least 65%. In some embodiments, the patient's glucose/creatinine ratio is decreased by at least 70%. In some embodiments, the patient's glucose/creatinine ratio is decreased by at least 75%. In some embodiments the patient's glucose/creatinine ratio is decreased by at least 80%. In some embodiments, the patient's glucose/creatinine ratio is decreased by at least 85%. In some embodiments, the patient's glucose/creatinine ratio is decreased by at least 90%. In some embodiments the patient's glucose/creatinine ratio is decrease ratio by at least 95%. In some embodiments, the patient's glucose/creatinine ratio is decreased ratio by 100%.

In some embodiments, the disclosure relates to methods of decreasing the patient's glucose/creatinine ratio, comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the glucose/creatinine ratio in the patient is decreased by at least 5 mg/mg (e.g., 5, 10, 15, 20, 25, 30, or 35 mg/mg). In some embodiments, the patient's glucose/creatinine ratio is decreased by at least 5 mg/mg. In some embodiments, the patient's glucose/creatinine ratio is decreased by at least 10 mg/mg. In some embodiments, the patient's glucose/creatinine ratio is decreased by at least 15 mg/mg. In some embodiments, the patient's glucose/creatinine ratio is decreased by at least 20 mg/mg. In some embodiments, the patient's glucose/creatinine ratio is decreased by at least 25 mg/mg. In some embodiments, the patient's glucose/creatinine ratio is decreased by at least 30 mg/mg. In some embodiments, the patient's glucose/creatinine ratio is decreased by at least 35 mg/mg.

### Complications of Hyperglycemia

High glucose concentrations in patients with hyperglycemia can cause injury to a large number of organs and tissues. The majority of cells in the body can adapt the rate of intracellular glucose transport under hyperglycemia conditions, and they can protect the intracellular milieu from the negative effect of high glucose levels. However, other cells, such as 3 cells, neuronal, and endothelial cells, are often unable to activate this control of glucose afflux and they equilibrate their intracellular glucose level to the extracellular concentrations, and therefore are more susceptible to the effect of hyperglycemia.

The dysregulation of glucose homeostasis in hyperglycemia can cause acute and chronic complications. Some of these complications include microvascular complications (e.g., retinopathy, nephropathy, and neuropathy), macrovascular complications (e.g., coronary artery disease, cerebrovascular disease, and peripheral vascular disease), endocrine emergencies (e.g., DKA and HHS), and vascular damage.

DKA is an acute life-threatening complication of diabetes, characterized by a patient having a combination of hyperglycemia (>250 mg/dl), metabolic acidosis (decreased pH and bicarbonates), and increased total body ketone concentration. HHS is a very serious acute hyperglycemic emergency which is diagnosed if patients have a glucose level >600 mg/dl and increased effective plasma osmolality >320 mOsm/kg, in the absence of ketoacidosis.

Certain conditions associated with acute hyperglycemia (e.g., increased renal perfusion, hyperfiltration, increased oxidative stress, decreased motor and sensory nerve conduction, increased collagen production in the kidney, and increased retinal perfusion) are known to lead to the development of microvascular complications. Microvascular complications are associated with short glucose excursions which may induce endothelial dysfunction, increase oxidative stress, activate coagulation factors, increase the expression of adhesion molecules, increase blood pressure, and dyslipidemia.

Chronic hyperglycemia can be evaluated by measuring hemoglobin A1c (HbA1c), a risk factor for the development of microvascular and macrovascular complications of hyperglycemia and diabetes. In some embodiments, acute hyperglycemia can be measured by evaluating fasting plasma glucose (FPG) or postprandial plasma glucose (PPG) levels. Glucose variability is an HbA1c-independent risk factor for the development of vascular complications, mainly in the context of type 2 diabetes. In some embodiments, glucose variability can be measured by evaluating the intraday glucose fluctuations from peaks to nadirs.

In certain aspects, the disclosure relates to methods of treating, preventing, or reducing the progression rate and/or severity of complications of hyperglycemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the one or more complications of hyperglycemia is selected from the group consisting of pre-diabetes mellitus, type 1 diabetes mellitus, type 2 diabetes mellitus, complications associated with diabetes mellitus, cataracts, microvascular disease, macrovascular disease, nephropathy, diabetic nephropathy, glomerulosclerosis, retinopathy, diabetic retinopathy, choroidal neovascularisation, coronary artery disease, cerebrovascular disease, peripheral vascular disease, DKA, hyperosmolar hyperglycemie state (HHS), NAFL disease, NASH, neuropathy, diabetic neuropathy, diabetic pain, tissue ischaemia, diabetic foot, diabetic ulcer, arteriosclerosis, hepatic steatosis, hypertension, and polycystic ovary syndrome. In some embodiments, the patient further has pulmonary hypertension. In some embodiments, the pulmonary hypertension is pulmonary arterial hypertension.

### Glucose Metabolism Disease, Prediabetes, and Diabetes

Glucose metabolism diseases includes a group of metabolic conditions that result in higher than normal blood glucose levels, such as prediabetes, diabetes mellitus, impaired fasting glucose (IFG), glucose intolerance, insulin resistance, and impaired glucose tolerance (IGT). IGT patients have two-hour glucose levels of 140 to 199 mg per dL (7.8 to 11.0 mmol) on the 75-g oral glucose tolerance test, and IFG patients have glucose levels of 100 to 125 mg per dL (5.6 to 6.9 mmol per L) when fasting. These glucose levels are above normal but below the level that is diagnostic for diabetes. Patients with IGT or IFG have a significant risk of developing diabetes and thus are an important target group for primary prevention.

In certain aspects, the disclosure relates to methods of treating, preventing, or reducing the progression rate and/or severity of glucose intolerance associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of glucose intolerance, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of glucose intolerance associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the patient further has pulmonary hypertension. In some embodiments, the pulmonary hypertension is pulmonary arterial hypertension.

In certain aspects, the disclosure relates to methods of treating, preventing, or reducing the progression rate and/or severity of a glucose metabolism disease associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of a glucose metabolism disease, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the disclosure relates to methods of treating, preventing, or reducing the progression rate and/or severity of a disorder associated with a glucose metabolism disease, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the glucose metabolism disease, disorder, or condition is a member selected from the group consisting of prediabetes, diabetes mellitus, impaired fasting glucose (IFG), glucose intolerance, insulin resistance, and impaired glucose tolerance (IGT). In some embodiments, the patient further has pulmonary hypertension. In some embodiments, the pulmonary hypertension is pulmonary arterial hypertension.

Prediabetes (also known as intermediate hyperglycemia) is a condition characterized by abnormal glucose concentrations which are below the cutoff for the diagnosis of diabetes. Prediabetes includes two main conditions: IFG, characterized by glucose levels between 100 and 125 mg/dl (5.6 to 6.9 mmol per L) in fasting patients, and IGT, defined by two-hour glucose levels of 140 to 199 mg per dL (7.8 to 11.0 mmol) on the 75-g oral glucose tolerance test.

In certain aspects, the disclosure relates to methods of treating, preventing, or reducing the progression rate and/or severity of prediabetes associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of prediabetes, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of prediabetes associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of prediabetes, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the patient further has pulmonary hypertension. In some embodiments, the pulmonary hypertension is pulmonary arterial hypertension.

Diabetes mellitus is a chronic condition characterized by hyperglycemia and alterations in protein and lipid metabolism. Diabetes patients typically have fasting glucose levels ≥126 mg/dl or random glucose levels ≥200 mg/dl. The most common form of diabetes is type 1 diabetes, which is characterized by autoimmune destruction of pancreatic β-cells, and is the most frequent form in the pediatric population, representing more than 90% of all cases of diabetes diagnosed during childhood and adolescence. The most common form of diabetes in adults is type 2 diabetes wherein most patients have insulin resistance associated with a progressive loss of β-cell function. Rates of type 2 diabetes in adolescents has been increasing with the growing epidemic of childhood obesity. Other forms of diabetes include secondary diabetes.

In certain aspects, the disclosure relates to methods of treating, preventing, or reducing the progression rate and/or severity of diabetes associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of diabetes, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of diabetes associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the one or more diabetes associated disorder is selected from the group consisting of: dyslipidemia, hyperlipidemia (total cholesterol level >240 mg/dL), hypercholesterolemia (e.g., total cholesterol level of >200 mg/dL, >220 mg/dL, >240 mg/dL, >250 mg/dL, or >275 mg/dL), low HDL serum level (e.g., <40 mg/dL, <45 mg/dL, or <50 mg/dL), high LDL serum level (e.g., ≥100 mg/dL, ≥130 mg/dL, ≥160 mg/dL, or ≥190 mg/dL), hypertriglyceridemia (e.g., a fasting TG level of ≥150 mg/dL, ≥175 mg/dL, ≥200 mg/dL, ≥300 mg/dL, ≥400 mg/dL, or ≥499 mg/dL), impaired glucose tolerance (IGT); maturity-onset diabetes of youth (MODY); leprechaunism (insulin receptor mutation), tropical diabetes, diabetes secondary to a pancreatic disease or surgery; diabetes associated with a genetic syndrome (e.g., Prader-Willi syndrome); pancreatitis; diabetes secondary to endocrinopathies; and adipositas. In some embodiments, the disclosure relates to methods of treating, preventing, or reducing the progression rate and/or severity of one or more complications of diabetes, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the one or more complications of diabetes is selected from the group consisting of DKA, vascular complications, hypertension, myocardial infarction, peripheral artery disease, vasoregulation dysfunction, arteriosclerosis, microvascular damage (e.g., retinopathy, nephropathy, and neuropathy), macrovascular damage, peripheral neuropathy, decreased vision, cataracts, blindness, cardiovascular disease, autonomic nervous system damage (e.g., decreased heart rate variability), liver disease, fatty liver disease, steatohepatitis, steatosis, cirrhosis, renal disease, and end-stage renal disease. In some instances, the subject has a body mass index BMI of 30 kg/m2 or greater (e.g., 30 to 39.9 kg/m2). In some embodiments, the patient has a BMI of at least 40 kg/m2. In some embodiments, the patient has central obesity (e.g., excess adiposity in the abdominal region, including belly fat and/or visceral fat). In some embodiments, the patient has a WHR of 0.85 or greater. In some embodiments, the patient has peripheral obesity (e.g., excess adiposity on the hips). In some embodiments, the ActRII antagonist treatment is an adjunct to diet and/or exercise. In some embodiments, the patient further has pulmonary hypertension. In some embodiments, the pulmonary hypertension is pulmonary arterial hypertension.

The diagnosis of type 2 diabetes can be determined based on blood glucose levels. For instance, a fasting plasma glucose level of 126 mg/dL or higher may indicative of type 2 diabetes. In some patients, a 2-hour plasma glucose level of 200 mg/dL or higher during a 75-g oral glucose tolerance test (OGTT) may be indicative of type 2 diabetes. In some patients, a random plasma glucose of 200 mg/dL or higher in the presence of symptoms of hyperglycemia is indicative of type 2 diabetes. In some embodiments, a hemoglobin A1c (HbA1c) level of 6.5% or higher is indicative of type 2 diabetes. In some embodiments, a patient with prediabetes or diabetes mellitus is further treated with a known diabetes therapy (e.g., diet changes, lifestyle changes, medications, and oral glucose-lowering agents)

### Hyperlipidemia, Dyslipidemia, and Arteriosclerosis

Hyperlipidemia can be characterized by a high level of total lipid content or level in a subject. Hyperlipidemia can also be accompanied by a high level of body weight or BMI of a subject. The types of lipid can include cholesterol, cholesterol esters, phospholipids and triglycerides. The content or level of the lipids can be a circulating level that is measured in the bloodstream, plasma or serum of the subject. These lipids can be transported in the blood as large lipoproteins including chylomicrons, very low-density lipoproteins (VLDL), intermediate-density lipoprotein (IDL), low-density lipoproteins (LDL) and high-density lipoproteins (HDL) based on their density. Most triglycerides can be transported in chylomicrons or VLDL and most cholesterol can be carried in LDL and HDL. High levels of lipid in the circulation can cause lipid accumulation on the walls of arteries, and further result in atherosclerotic plaque formation and therefore narrow the arteries. The subject that is suffering from hyperlipidemia can be at high risk of acquiring a cardiovascular condition. Hyperlipidemia can also be characterized by a high level of some lipoproteins or a low level of HDL. In some embodiments, the subject suffers from or is at risk of suffering from a condition that is associated with an abnormal level of lipoproteins or lipids in the subject. In some embodiments, the disclosure relates to methods for changing the levels of the one or more lipids or lipoproteins in the subject. In some embodiments, the one or more lipids or lipoproteins is selected from the group consisting of total cholesterol, triglyceride, HDL, IDL, VLDL and LDL.

In certain aspects, the disclosure relates to methods of treating, preventing, or reducing the progression rate and/or severity of hyperlipidemia associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of hyperlipidemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of hyperlipidemia associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of hyperlipidemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the patient further has pulmonary hypertension. In some embodiments, the pulmonary hypertension is pulmonary arterial hypertension.

In certain aspects, the disclosure relates to methods of treating, preventing, or reducing the progression rate and/or severity of dyslipidemia associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of dyslipidemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of dyslipidemia associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of dyslipidemia, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the patient further has pulmonary hypertension. In some embodiments, the pulmonary hypertension is pulmonary arterial hypertension. In some embodiments, the patient further has decreased renal function. In some embodiments, the method further improves renal function. In some embodiments, the subject has a total cholesterol level of >200 mg/dL, >220 mg/dL, >240 mg/dL, >250 mg/dL, or >275 mg/dL.

High levels of lipid in the circulation can cause lipid accumulation on the walls of arteries, and further result in atherosclerotic plaque formation and therefore narrow the arteries. Atherosclerosis is a specific type of arteriosclerosis. Atherosclerosis refers to the buildup of fats, cholesterol and other substances in and on the artery walls (plaque), which can restrict blood flow. In certain aspects, the disclosure relates to methods of treating, preventing, or reducing the progression rate and/or severity of arteriosclerosis associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of arteriosclerosis, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of arteriosclerosis associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of arteriosclerosis, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the arteriosclerosis is atherosclerosis. In some embodiments, the patient further has pulmonary hypertension. In some embodiments, the pulmonary hypertension is pulmonary arterial hypertension.

A number of methods can be used to assess the levels of lipoproteins and/or lipids in a subject. These methods can differ from one another in the type of sample and the analytical technique used. The type of sample that can be used to measure such levels include but are not limited to: serum, plasma, whole blood, red blood cells or tissue samples. Where desired, the level of lipoproteins and/or lipids can be measured under a fasting condition, e.g., without taking food for at least about 8 hours, 10 hours, 12 hours, or even longer.

In certain aspects, the disclosure relates to methods of reducing atherosclerotic plaque size associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of reducing atherosclerotic plaque size, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. The size of atherosclerotic plaque or lesion can be measured by any methods that are known in the art. In some embodiments, the patient further has pulmonary hypertension. In some embodiments, the pulmonary hypertension is pulmonary arterial hypertension.

### NAFLD and NASH

Nonalcoholic fatty liver disease (NAFLD) is a spectrum of increasingly common hepatic disorders characterized by fat accumulation in the liver (steatosis), often with deleterious effects. A subset of NAFLD patients develop an inflammatory condition referred to as nonalcoholic steatohepatitis (NASH), which can progress further to hepatic fibrosis, cirrhosis, and hepatocellular carcinoma.

In certain aspects, the disclosure relates to methods of treating, preventing, or reducing the progression rate and/or severity of NAFLD associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of NAFLD, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of NAFLD associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of NAFLD, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the NAFLD is NASH. In some embodiments, the patient further has pulmonary hypertension. In some embodiments, the pulmonary hypertension is pulmonary arterial hypertension.

In certain aspects, the disclosure relates to methods of treating, preventing, or reducing the progression rate and/or severity of NASH associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of NASH, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of NASH associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of NASH, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg. In some embodiments, the one or more complications of NASH is selected from the group consisting of weakness, fatigue, unexplained weight loss, ache and jaundice. In some embodiments, the patient further has pulmonary hypertension. In some embodiments, the pulmonary hypertension is pulmonary arterial hypertension.

### Fibrosis

Fibrosis generally refers to an excessive deposition of both collagen fibers and extracellular matrix combined with a relative decrease of cell number in an organ or tissue. While this process is an important feature of natural wound healing following injury, fibrosis can lead to pathological damage in various tissue and organs including, for example, the lungs, kidneys, liver, bone, muscle, and skin. The role TGF-beta in fibrosis has been extensively study. However, other TGF-beta superfamily ligands have also been implicated in fibrosis including, for example, activins (e.g., activin A and activin B) and GDF8 [Hedger et al (2013) Cytokine and Growth Factor Reviews 24:285-295; Hardy et al. (2015) 93: 567-574; and Cantini et al. (2008) J Sex Med 5:1607-1622]. Therefore, in some instances, an ActRII polypeptide (e.g., an amino acid sequence that is at least 90% identical to an amino acid sequence corresponding to residues 30-110 of SEQ ID NO: 1), or combinations of such antagonists, of the present disclosure can be used to treat fibrosis, particularly fibrosis-associated disorders and conditions. In certain aspects, the disclosure relates to methods of treating, preventing, or reducing the progression rate and/or severity of fibrosis associated with pulmonary hypertension comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of fibrosis, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of fibrosis associated disorders, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure relates to methods of treating, preventing, or reducing the progression rate and/or severity of one or more complications of fibrosis, comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the polypeptide is administered at a dosing range of 0.1 mg/kg to 2.0 mg/kg.

In some embodiments, the fibrosis is selected from the group consisting of pulmonary fibrosis, hypersensitivity pneumonitis, idiopathic fibrosis, fibrosis associated with tuberculosis, fibrosis associated with pneumonia, cystic fibrosis, fibrosis associated with asthma, fibrosis associated with chronic obstructive pulmonary disease (COPD), fibrosis associated with emphysema, renal fibrosis, fibrosis associated with renal failure, fibrosis associated with chronic renal disease, bone fibrosis, myelofibrosis, fibrosis associated with rheumatoid arthritis, fibrosis associated with systemic lupus erythematosus, fibrosis associated with scleroderma, fibrosis associated with sarcoidosis, fibrosis associated with granulomatosis with polyangiitis, fibrosis associated with Peyronie's disease, liver fibrosis, fibrosis associated with Wilson's disease, fibrosis associated with glycogen storage diseases (particularly types III, IV, IX, and X), fibrosis associated with Gaucher disease, fibrosis associated with Zellweger syndrome, fibrosis associated with biliary cirrhosis, fibrosis associated with sclerosing cholangitis, fibrosis associated with Budd-Chiari syndrome, surgery-associated fibrosis, fibrosis associated with Crohn's disease, fibrosis associated with Duputren's contracture, mediastinal fibrosis, nephrogeneic fibrosis, retroperitoneal fibrosis, atrial fibrosis, endomyocardial fibrosis, and pancreatic fibrosis. In some embodiments, the patient further has pulmonary hypertension. In some embodiments, the pulmonary hypertension is pulmonary arterial hypertension.

### Pulmonary Hypertension

The disclosure relates to methods of treating, preventing, or reducing the progression rate and/or severity of a disorder associated with pulmonary hypertension. For instance, a disorder associated with pulmonary hypertension includes any disorder selected from the group consisting of hyperglycemia, insulin sensitivity, insulin resistance, glucose metabolism disease, hyperlipidemia, dyslipidemia, arteriosclerosis, NAFLD, NASH, fibrosis, prediabetes, diabetes mellitus, type 1 diabetes mellitus, type 2 diabetes mellitus, and complications thereof. A pulmonary hypertension condition which may be associated with the disorders treated by methods described herein, can comprise any one or more of the conditions recognized according to the World Health Organization (WHO). See, e.g., Simonneau (2019) Eur Respir J: 53:1801913.

TABLE 1
Clinical Classification of Pulmonary Hypertension

Group 1: Pulmonary arterial hypertension (PAH)
1.1 Idiopathic PAH
1.2 Heritable PAH
1.2.1 BMPR2
1.2.2 ALK-1, ENG, SMAD9, CAV1, KCNK3
1.2.3 Unknown
1.3 Drug and toxin induced PAH
1.4 Associated with:
1.4.1 Connective tissue disease
1.4.2 HIV infection
1.4.3 Portal hypertension
1.4.4 Congenital heart diseases
1.4.5 Schistosomiasis
1.5 PAH long-term responders to calcium channel blockers
1.6 PAH with overt features of venous/capillaries (PVOD/PCH) involvement
1.7 Persistent PH of the newborn syndrome
Group 2: Pulmonary hypertension due to left heart disease
2.1 PH due to heart failure with preserved LVEF¹ (HFpEF)
2.2 PH due to heart failure with reduced LVEF (HFrEF)
2.3 Valvular heart disease
2.4 Congenital/acquired cardiovascular conditions leading to post-capillary PH
Group 3: Pulmonary hypertension due to lung disease and/or hypoxia
3.1 Obstructive lung disease
3.2 Restrictive lung disease
3.3 Other lung disease with mixed restrictive/obstructive pattern
3.4 Hypoxia without lung disease
3.5 Developmental lung disorders
Group 4: Pulmonary hypertension due to pulmonary artery obstructions
4.1 Chronic thromboembolic PH
4.2 Other pulmonary artery obstructions
4.2.1 Sarcoma (high or intermediate grade) or angiosarcoma
4.2.2 Other malignant tumours
Renal carcinoma
Uterine carcinoma
Germ cell tumours of the testis
Other tumours
4.2.3 Non-malignant tumours
Uterine leiomyoma
4.2.4 Arteritis without connective tissue disease
4.2.5 Congenital pulmonary artery stenoses
4.2.6 Parasites
Hydatidosis
Group 5: Pulmonary hypertension with unclear and/or multifactorial mechanisms.
5.1 Hematological disorders (e.g., Chronic hemolytic anaemia and myeloproliferative
disorders)
5.2 Systemic and metabolic disorders (e.g., Pulmonary Langerhans cell histiocytosis,
Gaucher disease, Glycogen storage disease, Neurofibromatosis, and Sarcoidosis)
5.3 Others (e.g., Chronic renal failure with or without haemodialysis and Fibrosing
mediastinitis)
5.4 Complex congenital heart disease
¹Left ventricular ejection fraction

The clinical purpose of the classification of PH is to categorize clinical conditions associated with PH into five groups according to their pathophysiological mechanisms, clinical presentation, hemodynamic characteristics, and treatment strategy. This clinical classification may be updated when new data are available on the above features or when additional clinical entities are considered.

Pulmonary hypertension (PH) has been previously classified as primary or secondary PH. The term primary pulmonary hypertension has now been replaced by idiopathic PAH or familial PAH depending on the absence or presence of genetic information; the term secondary pulmonary hypertension has been abandoned.

As used herein, the term “pulmonary hemodynamic parameter” refers to any parameter used to describe or evaluate the blood flow through the heart and pulmonary vasculature. Examples of pulmonary hemodynamic parameters include, but are not limited to, mean pulmonary artery pressure (mPAP), diastolic pulmonary artery pressure (dPAP) [also known as pulmonary artery diastolic pressure (PADP)], systolic pulmonary artery pressure (sPAP) [also known as pulmonary artery systolic pressure (PASP)], pulmonary capillary wedge pressure (PCWP) [also known as pulmonary artery wedge pressure (PAWP)], left ventricular end-diastolic pressure (LVEDP), diastolic pressure gradient (DPG) [also known as diastolic pressure difference (DPD)], left atrial pressure (LAP), transpulmonary gradient (TPG), pulmonary vascular resistance (PVR) and cardiac output (CO).

Many of the pulmonary hemodynamic parameters described above are interrelated. For example, PCWP is often used as a more convenient, less invasive approximation of LAP.

As another example, PVR is related to mPAP, PCWP and CO according to the following equation:

PVR=(mPAP−PCWP)/CO[Woods Units]

The PVR measures the resistance to flow imposed by the pulmonary vasculature without the influence of the left-sided filling pressure. PVR can also be measured according to the following equations:

PVR=TPG×80/CO[unit: dynes-sec-cm⁻⁵] OR PVR=(mPAP−PCWP)×80/CO [unit: dynes-sec-cm⁻⁵]

According to some embodiments, a pre-capillary pulmonary arterial contribution to PH may be reflected by an elevated PVR. In some embodiments, the normal PVR is 20-130 dynes-sec-cm⁻⁵ or 0.5-1.1 Wood units. According to some embodiments, an elevated PVR may refer to a PVR above 2 Wood units, above 2.5 Wood units, above 3 Wood units or above 3.5 Wood units.

As yet another example, TPG is the difference between mPAP and left atrial pressure (PLA; commonly estimated by pulmonary capillary wedge pressure: PCWP) as shown by the following equation: TPG-mPAP-PCWP

The TPG is influenced by all the determinants of mPAP, including flow, resistance and left heart filling pressure. A pre-capillary pulmonary arterial contribution to PH may be reflected by an increased trans-pulmonary gradient (TPG). According to some embodiments, an increased TPG may refer to an mPAP-PCWP that exceeds 12-15 mmHg.

DPG (defined as diastolic PAP—mean PAWP) appears to best approach the characteristics required to determine pulmonary vascular disease. In some embodiments, the DPG is synonymous with diastolic pressure difference (DPD). In normal subjects, DPG generally lies in the 1-3 mmHg range, and in patients evaluated for cardiac disease (excluding shunts), DPG remains ≤5 mmHg in most cases.

As a further example, mPAP is related to dPAP and sPAP according to the following equation: mPAP=(⅔)dPAP+(⅓)sPAP

In some embodiments, the pulmonary hemodynamic parameters are measured directly, such as during a right heart catheterization. In other embodiments, the pulmonary hemodynamic parameters are estimated and/or evaluated through other techniques such as magnetic resonance imaging (MRI) or echocardiography.

Exemplary pulmonary hemodynamic parameters include mPAP, PAWP, TPG, DPG, and PVR. The one or more pulmonary hemodynamic parameters may be measured by any appropriate procedures, such as by utilizing a right heart catheterization or echocardiography. Various hemodynamic types of PH are shown in Table 2 together with their corresponding clinical classification (Table 1).

TABLE 2
Hemodynamic Types of Pulmonary Hypertension (PH)

| Hemodynamic | Hemodynamic | | WHO PH |
|---|---|---|---|
| Type | Subtype | Characteristics | Classification |
| Pulmonary | — | mPAP >20 mmHg | All (Groups 1-5) |
| Hypertension | | | |
| Pre-Capillary PH | — | mPAP >20 mmHg | Group 1: Pulmonary |
| | | PAWP ≤15 mmHg | arterial hypertension |
| | | PVR ≥3 Wood units | Group 3: PH due to |
| | | | lung disease and/or |
| | | | hypoxia |
| | | | Group 4: PH due to |
| | | | pulmonary artery |
| | | | obstructions |
| | | | Group 5: PH with |
| | | | unclear and/or |
| | | | multifactorial |
| | | | mechanisms. |
| Post-Capillary | Isolated Post- | mPAP >20 mmHg | Group 2: PH due to |
| PH | Capillary PH | PAWP >15 mmHg | left heart disease |
| | | PVR <3 Wood units | Group 5: PH with |
| | | DPG <7 mmHg | unclear and/or |
| | Combined Pre- | mPAP >20 mmHg | multifactorial |
| | and Post- | PAWP >15 mmHg | mechanisms. |
| | Capillary PH | PVR ≥3 Wood units | |
| | | DPG ≥7 mmHg | |

The types of PH and the difference between pre-capillary pulmonary hypertension and post-capillary pulmonary hypertension are based on pulmonary hemodynamic parameters. As used herein, the term “pre-capillary pulmonary hypertension” includes WHO clinical Groups 1, 3, 4, and 5. In general, pre-capillary pulmonary hypertension is characterized using the pulmonary hemodynamic parameters shown in Table 2 (i.e., an mPAP>20 mmflg or in some embodiments a mPAP>25 mmflg). As used herein, the term “post-capillary pulmonary hypertension” (PcPH) includes both isolated post-capillary pulmonary hypertension (IpcPH) and combined pre- and post-capillary pulmonary hypertension (CpcPH), both within WHO clinical Groups 2 and 5. In some embodiments, IpcPH is characterized using the pulmonary hemodynamic parameters shown in Table 2 (i.e., one or more of the following pulmonary hemodynamic parameters: mPAP>20 mmHg, PAWP>15 mmHg, PVR<3 Wood units, and/or DPG<7 mmHg). In some embodiments, CpcPH is characterized using the pulmonary hemodynamic parameters shown in Table 2 (i.e., one or more of the following pulmonary hemodynamic parameters: mPAP>20 mmHg, PAWP>15 mmHg, PVR≥3 Wood units, and/or DPG≥7 mmHg).

The clinical classification or hemodynamic type of PH described herein and the associated diagnostic parameters may be updated or varied based on the availability of new or existing sources of data or when additional clinical entities are considered.

### Diagnosis of PH

The diagnosis of PH, including WHO PH class and functional group, can be determined based on symptoms and physical examination using a review of a comprehensive set of parameters to determine if the hemodynamic and other criteria are met. Some of the criteria which may considered include the patient's clinical presentation (e.g., shortness of breath, fatigue, weakness, angina, syncope, dry-couch, exercise-induced nausea and vomiting), electrocardiogram (ECG) results, chest radiograph results, pulmonary function tests, arterial blood gases, echocardiography results, ventilation/perfusion lung scan results, high-resolution computed tomography results, contrast-enhanced computed tomography results, pulmonary angiography results, cardiac magnetic resonance imaging, blood tests (e.g., biomarkers such as BNP or NT-proBNP), immunology, abdominal ultrasound scan, right heart catherization (RHC), vasoreactivity, and genetic testing. See, e.g., Galie N., et al Euro Heart J. (2016) 37, 67-119.

In some embodiments, a biomarker may be used to determine the diagnosis of PH. For instance, in some embodiments, the biomarker is a marker of vascular dysfunction (e.g., asymmetric dimethylarginine (ADMA), endothelin-1, angiopoeitins, or von Willebrand factor). In some embodiments, the biomarker is a marker of inflammation (C-reactive protein, interleukin 6, chemokines). In some embodiments, the biomarker is a marker of myocardial stress (e.g., (atrial natriuretic peptide, brain natriuretic peptide (BNP)/NT-proBNP, or troponins). In some embodiments, the biomarker is a marker of low CO and/or tissue hypoxia (e.g., pCO2, uric acid, growth differentiation factor 15 (GDF15), or osteopontin). In some embodiments, the biomarker is a marker of secondary organ damage (e.g., creatinine or bilirubin). See, e.g., Galie N., et al Euro Heart J. (2016) 37, 67-119.

### Group 1 PH

Pulmonary arterial hypertension (WHO Group 1 PH) is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in PVR and sustained elevations in mPAP, which ultimately lead to right ventricular failure and death. Patients diagnosed with PAH have a poor prognosis and equally compromised quality of life, with a mean life expectancy of 2 to 5 years from the time of diagnosis if untreated.

PAH can be diagnosed based on an increase in blood pressure in the pulmonary artery mean pulmonary arterial pressure above 25 mmHg (or above 20 mmHg under updated guidelines) at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, ENG, SMAD9, CAV1, KCNK3, or EIF2AK4). In 70% of familial PAH cases, mutations are located in the BMPR2 gene. Risk factors for the development of PAH include family history of PAH, drug and toxin use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus). PAH may be associated with long term responders to calcium channel blockers, overt features of venous/capillaries (PVOD/PCH) involvement, and persistent PH of the newborn syndrome.

### Group 2 PH

Pulmonary hypertension due to left heart disease (PH-LHD) (WHO Group 2 PH) is a complex pathophenotype that, when present, may result in an increased susceptibility to adverse events and worse clinical outcome. PH-LHD is sometimes defined as patients having a pulmonary capillary wedge pressure (PCWP)>15 mmHg and a mean pulmonary artery pressure (mPAP)≥25 mmHg (or a mean pulmonary artery pressure (mPAP)≥20 mmHg under updated guidelines). PH-LHD occurs as a consequence of the backward transmission of high left sided filling pressures, mainly driven by LV diastolic function, directly to the post-capillary pulmonary vessels and, thereby, to the rest of the pulmonary circulation. PH-LHD may be associated with or caused by PH due to heart failure with preserved left ventricle ejection fraction (LVEF) (also known as HFpEF), PH due to heart failure with reduced LVEF (also known as HFrEF), valvular heart disease, or congenital/acquired cardiovascular conditions leading to post-capillary PH. Compared with PAH, patients with PH-LHD are often older, female, with a higher prevalence of cardiovascular co-morbidities and most, if not all, of the features of metabolic syndrome.

Among those patients with PH-LHD, two phenotypes have been described: 1) a group of isolated post-capillary (IpcPH) or “passive” PH in which elevated pulmonary pressures are reversible and in proportion to increases in left atrial pressure, and 2) a group with an added “pre-capillary” component [combined post-capillary and pre-capillary PH (CpcPH)]. This latter group, CpcPH, may have comorbid pulmonary vascular remodeling and therefore may demonstrate persistent PH after interventions to lower left sided filling pressures.

In some embodiments, a combination of mPAP, PAWP, PVR, or DPG may be used to define the different subtypes of PH-LHD, i.e., IpcPH and CpcPH (see, e.g., Table 2). In some embodiments, patients with CpcPH are characterized as having a TPG>12-15 mmHg and a PVR>2.5-3 Wood units (WU). In some embodiments, CpcPH is distinguished from IpcPH using the DPG. In some embodiments, a patient with CpcPH has a DPG≥7 mmHg. In some embodiments, a patient with IpcPH has a DPG<7 mmHg.

In some embodiments, a combination of DPG and PVR may be used to define the different types of PH-LHD. For instance, in some embodiments, IpcPH patients have a DPG<7 mmHg and/or a PVR of ≤3 WU. In some embodiments, CpcPH patients have a DPG≥7 mmHg and/or a PVR>3 WU.

The clinical classification or hematological classification described herein and the associated diagnostic parameters may be updated when new data are available or when additional clinical entities are considered. For instance, at the 5^{th} World Symposium on Pulmonary Hypertension (WSPH), a new terminology was adopted to distinguish IpcPH from CpcPH, based on the diastolic pressure difference/gradient (DPG) between the dPAP and PAWP. However, this definition was found to be too restrictive and exposed to interpretation, leading to controversies about whether the DPG would or would not predict outcome in patients with Group 2 PH. Accordingly, at the 6′ WSPH, pulmonary vascular resistance (PVR) was subsequently reintroduced to better reflect the impact of the right ventricle on patient outcome. See, e.g., Vachiery J. L., et al. Eur Respir J 2019 Jan. 24; 53(1).

Therapies for treating PH-LHD primarily include treatment of the underlying condition (i.e., COPD, sleep apnea syndrome, CTEPH) prior to considering specific measures to treat the PH itself. Some therapies include repair of valvular heart disease (if indicated). Non-specific vasodilators such as nitrates and hydralazine may also be used. In some embodiments, an LV assist device (LVAD) may be used to lower pulmonary pressure. The lack of specific therapies is particularly problematic because PH-LHD is the most common cause of PH in western countries and its presence commonly results in adverse course of the disease. Specifically, the presence of PH-LHD can result in more severe symptoms in LHD, worse exercise tolerance, and a negative impact on outcome.

### Group 3 PH

Pulmonary hypertension due to lung disease and/or hypoxia (WHO Group 3 PH) refers to a form of pulmonary hypertension that is due to lung disease or chronic hypoxia. This form of PH is also known as “hypoxic PH” or “hypoxic pulmonary hypertension.” Hypoxic PH may be associated with or caused by chronic obstructive pulmonary disease (e.g., emphysema), interstitial lung disease, sleep-disordered breathing (e.g., sleep apnea), lung disease (e.g., pulmonary fibrosis), alveolar hypoventilation disorders, chronic exposure to high altitude, or developmental abnormalities.

### Group 4 PH

Pulmonary hypertension due to pulmonary artery obstructions (WHO Group 4 PH) is a form of pulmonary hypertension that is related to chronic arterial obstruction (e.g., blood clots). There may be multiple pathophysiological mechanisms driving development of PH in Group 4 including chronic thromboembolic PH, sarcoma (high or intermediate grade) or angiosarcoma, other malignant tumors (e.g., renal carcinoma, uterine carcinoma, germ cell tumors of the testis, or other tumors), non-malignant tumors (e.g., uterine leiomyoma), arteritis without connective tissue disease, congenital pulmonary artery stenosis, or parasites (e.g., hydatidosis).

Various pulmonary hemodynamic parameters are associated with Group 4 PH. For instance, in patients with PH due to pulmonary artery obstructions, those with severe PH (>40 mmHg) often have a marked increase in PVR (around 10 WU); more often these patients may have a mild PH (mPAP 20-30 mmHg), associated with lower PVR but remaining generally >3 WU. See, e.g., Simonneau (2019) Eur Respir J: 53:1801913. In these different chronic lung diseases, even a modest elevation in mPAP (20-29 mmHg) can be associated with a poor prognosis. Furthermore, in chronic thromboembolism, patients may have severe pre-capillary PH with a mPAP of about 47 mmHg and a mean PVR of about 8.9 WU. Id. In this setting, even in patients with mild elevation of mPAP (20-24 mmHg), PVR is generally >3 WU.

### Group 5 PH

Pulmonary hypertension with unclear and/or multifactorial mechanisms (WHO Group 5 PH) is a group which contains less-studied forms of PH in comparison with the other groups. However, many of the PH forms currently in group 5 represent a significant part of the PH burden. The diseases within Group 5 PH are characterized by having no identified predominant mechanism driving the development of PH. There may be multiple pathophysiological mechanisms driving development of PH, including hematological disorders (e.g., chronic hemolytic anemia or myeloproliferative disorders), systemic and metabolic disorders (e.g., Pulmonary Langerhans cell histiocytosis, Gaucher disease, glycogen storage disease, neurofibromatosis, or sarcoidosis), others (e.g., chronic renal failure with or without hemodialysis or fibrosing mediastinitis), or complex congenital heart disease.

In certain aspects, the disclosure relates to methods of treating, preventing, or reducing the progression rate and/or severity of a disease (e.g., hyperglycemia) associated with pulmonary hypertension (e.g., pulmonary arterial hypertension) comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the pulmonary hypertension is PcPH. In some embodiments, the pulmonary hypertension is IpcPH. In certain embodiments, the pulmonary hypertension is CpcPH. In some embodiments, the pulmonary hypertension is Group 2 PH as classified by the WHO. In some embodiments, the pulmonary hypertension is pulmonary hypertension due to heart failure with preserved LVEF (HFpEF). In some embodiments, the pulmonary hypertension is pulmonary hypertension due to heart failure with reduced LVEF (HFrEF). In some embodiments, the pulmonary hypertension is valvular heart disease. In some embodiments, the pulmonary hypertension is congenital/acquired cardiovascular conditions leading to post-capillary PH. In some embodiments, the pulmonary hypertension is pulmonary hypertension with unclear and/or multifactorial mechanisms. In some embodiments, the pulmonary hypertension is Group 5 PH as classified by the WHO.

In some embodiments, the disclosure relates to methods of treating, preventing, or reducing the progression rate and/or severity of a disease (e.g., hyperglycemia) associated with pulmonary hypertension, wherein the pulmonary hypertension comprises combinations of certain patient populations. Each of the patient populations described herein can be combined and reorganized accordingly. For instance, in some embodiments, the pulmonary hypertension is CpcPH patients who have PH due to HFpEF. In some embodiments, the pulmonary hypertension is CpcPH patients who have PH due to heart failure with reduced LVEF (HFrEF). In some embodiments, the pulmonary hypertension is IpcPH patients who have PH due to heart failure with preserved LVEF (HFpEF). In some embodiments, the pulmonary hypertension is IpcPH patients who have PH due to HFrEF.

In some embodiments, the method relates to patients that have a disease associated with pulmonary hypertension and pulmonary hypertension with unclear and/or multifactorial mechanisms. In some embodiments, the method relates to patients that have a disease associated with pulmonary hypertension and a hematological disorder (e.g., chronic hemolytic anemia and myeloproliferative disorders). In some embodiments, the method relates to patients that have a disease associated with pulmonary hypertension and a systemic and/or metabolic disorder (e.g., pulmonary Langerhans cell histiocytosis, Gaucher disease, glycogen storage disease, neurofibromatosis, and sarcoidosis). In some embodiments, the method relates to patients that have a disease associated with pulmonary hypertension and other disorders with unclear and/or multifactorial mechanisms (e.g., chronic renal failure with or without hemodialysis or fibrosing mediastinitis). In some embodiments, the method relates to patients that have a disease associated with pulmonary hypertension and complex congenital heart disease.

Optionally, methods disclosed herein for treating a disorder associated with pulmonary hypertension (e.g., hyperglycemia), may further comprise administering to the patient one or more supportive therapies or additional active agents for treating pulmonary hypertension. For example, the patient also may be administered one or more supportive therapies or active agents selected from the group consisting of: nitrates, hydralazine, prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, darusentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat, vericiguat, and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1->3)-beta-D-glucopyranosyl] oleanolic acid; 3-O-[beta-D-glucopyranosyl-(1->2)-beta-D-glucopyranosyl] oleanolic acid; 3-O-[beta-D-glucopyranosyl-(1->3)-beta-D-glucopyranosyl] oleanolic acid 28-O-beta-D-glucopyranosyl ester; 3-O-[beta-D-glucopyranosyl-(1->2)-beta-D-glucopyranosyl] oleanolic acid 28-O-beta-D-glucopyranosyl ester; 3-O-[α-L-rhamnopyranosyl-(1->3)-beta-D-glucuronopyranosyl]oleanolic acid; 3-O-[alpha-L-rhamnopyranosyl-(1->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate), an LV assist device (LVAD), lung and/or heart transplantation.

In some embodiments, the methods described herein may further comprise administering to the patient an angiotensin antagonist (e.g., angiotensin receptor blocker, ARB). In some embodiments, the patient is further administered one or more ARBs selected from the group consisting of losartan, irbesartan, olmesartan, candesartan, valsartan, fimasartan, azilsartan, salprisartan, and telmisartan. In some embodiments, the patient is administered losartan. In some embodiments, the patient is administered irbesartan. In some embodiments, a patient is administered olmesartan. In some embodiments, the patient is administered candesartan. In some embodiments, the patient is administered valsartan. In some embodiments, the patient is administered fimasartan. In some embodiments, the patient is administered azilsartan. In some embodiments, the patient is administered salprisartan. In some embodiments, a patient is administered telmisartan.

In some embodiments, the methods described herein may further comprise administering to the patient one or more ACE inhibitors. In some embodiments, the one or more ACE inhibitors are selected from the group consisting of benazepril, captopril, enalapril, lisinopril, perindopril, ramipril (e.g., ramipen), trandolapril, and zofenopril. In some embodiments, a patient is administered benazepril. In some embodiments, the patient is administered captopril. In some embodiments, the patient is administered enalapril. In some embodiments, the patient is administered lisinopril. In some embodiments, a patient is administered perindopril. In some embodiments, the patient is administered ramipril. In some embodiments, the patient is administered trandolapril. In some embodiments, the patient is administered zofenopril. In some embodiments, the methods described herein may further comprise administering to the patient an ARB and an ACE inhibitor. In some embodiments, an alternative approach to angiotensin antagonism is to combine an ACE inhibitor and/or ARB with an aldosterone antagonist.

In some embodiments, the one or more supportive therapies or additional active agents for treating pulmonary hypertension are administered prior to administration of the ActRII polypeptide. In some embodiments, the one or more supportive therapies or additional active agents for treating pulmonary hypertension are administered in combination with the ActRII polypeptide. In some embodiments, the one or more supportive therapies or additional active agents for treating pulmonary hypertension are administered after the administration of the ActRII polypeptide. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

### Measuring Hematologic Parameters in a Patient

In certain embodiments, the present disclosure provides methods for managing a patient that has been treated with, or is a candidate to be treated with, one or more one or more ActRII polypeptides of the disclosure (e.g., an amino acid sequence that is at least 90% identical to an amino acid sequence corresponding to residues 30-110 of SEQ ID NO: 1) by measuring one or more hematologic parameters in the patient. The hematologic parameters may be used to evaluate appropriate dosing for a patient who is a candidate to be treated with one or more ActRII polypeptides of the present disclosure, to monitor the hematologic parameters during treatment, to evaluate whether to adjust the dosage during treatment with one or more ActRII polypeptides of the disclosure, and/or to evaluate an appropriate maintenance dose of one or more ActRII polypeptides of the disclosure. If one or more of the hematologic parameters are outside the normal level, dosing with one or more ActRII polypeptides may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more ActRII polypeptides, then onset of administration of the one or more ActRII polypeptides of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels, then administration of the one or more ActRII polypeptides of the disclosure may be delayed until the levels have returned to a normal or acceptable level.

In certain embodiments, if one or more hematologic parameters are outside the normal range or on the high side of normal in a patient who is a candidate to be treated with one or more ActRII polypeptides, then the onset of administration may not be delayed. However, the dosage amount or frequency of dosing of the one or more ActRII polypeptides of the disclosure may be set at an amount that would reduce the risk of an unacceptable increase in the hematologic parameters arising upon administration of the one or more ActRII polypeptides of the disclosure. Alternatively, a therapeutic regimen may be developed for the patient that combines one or more ActRII polypeptides with a therapeutic agent that addresses the undesirable level of the hematologic parameter. For example, if the patient has elevated blood pressure, then a therapeutic regimen may be designed involving administration of one or more ActRII polypeptides (e.g., an amino acid sequence that is at least 90% identical to an amino acid sequence corresponding to residues 30-110 of SEQ ID NO: 1) and a blood pressure lowering agent. For a patient having lower than desired iron stores, a therapeutic regimen may be developed involving one or more ActRII polypeptides of the disclosure and iron supplementation.

In one embodiment, baseline parameter(s) for one or more hematologic parameters may be established for a patient who is a candidate to be treated with one or more ActRII polypeptides of the disclosure and an appropriate dosing regimen established for that patient based on the baseline value(s). Alternatively, established baseline parameters based on a patient's medical history could be used to inform an appropriate ActRII polypeptide dosing regimen for a patient. For example, if a healthy patient has an established baseline blood pressure reading that is above the defined normal range it may not be necessary to bring the patient's blood pressure into the range that is considered normal for the general population prior to treatment with the one or more ActRII polypeptides of the disclosure. A patient's baseline values for one or more hematologic parameters prior to treatment with one or more ActRII polypeptides of the disclosure may also be used as the relevant comparative values for monitoring any changes to the hematologic parameters during treatment with the one or more ActRII polypeptides of the disclosure.

In certain embodiments, one or more hematologic parameters are measured in patients who are being treated with one or more ActRII polypeptides. The hematologic parameters may be used to monitor the patient during treatment and permit adjustment or termination of the dosing with the one or more ActRII polypeptides of the disclosure or additional dosing with another therapeutic agent. For example, if administration of one or more ActRII polypeptides results in an increase in blood pressure, red blood cell level, or hemoglobin level, or a reduction in iron stores, then the dose of the one or more ActRII polypeptides of the disclosure may be reduced in amount or frequency in order to decrease the effects of the one or more ActRII polypeptides of the disclosure on the one or more hematologic parameters. If administration of one or more ActRII polypeptides results in a change in one or more hematologic parameters that is adverse to the patient, then the dosing of the one or more ActRII polypeptides of the disclosure may be terminated either temporarily, until the hematologic parameter(s) return to an acceptable level, or permanently. Similarly, if one or more hematologic parameters are not brought within an acceptable range after reducing the dose or frequency of administration of the one or more ActRII polypeptides of the disclosure, then the dosing may be terminated. As an alternative, or in addition to, reducing or terminating the dosing with the one or more ActRII polypeptides of the disclosure, the patient may be dosed with an additional therapeutic agent that addresses the undesirable level in the hematologic parameter(s), such as, for example, a blood pressure lowering agent or an iron supplement. For example, if a patient being treated with one or more ActRII polypeptides has elevated blood pressure, then dosing with the one or more ActRII polypeptides of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the patient may be treated with a blood-pressure-lowering agent.

### 5. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII polypeptides which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., ActRII polypeptides) to a target tissue site, providing a structure for the developing tissue and optimally capable of being resorbed into the body. For example, the matrix may provide slow release of the ActRII polypeptide. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII polypeptides). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling. In some embodiments, ActRII polypeptides of the disclosure are administered at 0.1 mg/kg. In some embodiments, ActRII polypeptides of the disclosure are administered at 0.2 mg/kg. In some embodiments, ActRII polypeptides of the disclosure are administered at 0.3 mg/kg. In some embodiments, ActRII polypeptides of the disclosure are administered at 0.4 mg/kg. In some embodiments, ActRII polypeptides of the disclosure are administered at 0.5 mg/kg. In some embodiments, ActRII polypeptides of the disclosure are administered at 0.6 mg/kg. In some embodiments, ActRII polypeptides of the disclosure are administered at 0.7 mg/kg. In some embodiments, ActRII polypeptides of the disclosure are administered at 0.8 mg/kg. In some embodiments, ActRII polypeptides of the disclosure are administered at 0.9 mg/kg. In some embodiments, ActRII polypeptides of the disclosure are administered at 1.0 mg/kg. In some embodiments, ActRII polypeptides of the disclosure are administered at 1.1 mg/kg. In some embodiments, ActRII polypeptides of the disclosure are administered at 1.2 mg/kg. In some embodiments, ActRII polypeptides of the disclosure are administered at 1.3 mg/kg. In some embodiments, ActRII polypeptides of the disclosure are administered at 1.4 mg/kg. In some embodiments, ActRII polypeptides of the disclosure are administered at 1.5 mg/kg. In some embodiments, ActRII polypeptides of the disclosure are administered at 1.6 mg/kg. In some embodiments, ActRII polypeptides of the disclosure are administered at 1.7 mg/kg. In some embodiments, ActRII polypeptides of the disclosure are administered at 1.8 mg/kg. In some embodiments, ActRII polypeptides of the disclosure are administered at 1.9 mg/kg. In some embodiments, ActRII polypeptides of the disclosure are administered at 2.0 mg/kg.

In certain embodiments, the present invention also provides gene therapy for the in vivo production of ActRII polypeptides. Such therapy would achieve its therapeutic effect by introduction of the ActRII polypeptide polynucleotide sequences into cells or tissues having the disorders as listed above. Delivery of ActRII polypeptide polynucleotide sequences can be achieved using a recombinant expression vector such as a chimeric virus or a colloidal dispersion system. Preferred for therapeutic delivery of ActRII polypeptide polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the ActRII polypeptide. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII polypeptide polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### 6. Kits

The present disclosure provides a kit comprising a lyophilized polypeptide and an injection device. In certain embodiments, the lyophilized polypeptide comprises an ActRII polypeptide (e.g., a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1), or fragments, functional variants, or modified forms thereof. In certain embodiments, the lyophilized polypeptide binds to one or more ligands selected from the group consisting of activin A, activin B, and GDF11. In certain such embodiments, the lyophilized polypeptide further binds to one or more ligands selected from the group consisting of BMP10, GDF8, and BMP6. In certain embodiments, the lyophilized polypeptide binds to activin and/or GDF11.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of a polypeptide beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 1. In certain such embodiments, the polypeptide comprises an amino acid sequence that is least 90%, 95%, or 99% identical to an amino acid sequence corresponding to residues 30-110 of SEQ ID NO: 1, wherein the polypeptide binds to activin and/or GDF11. In certain embodiments, the polypeptide comprises the amino acid sequence corresponding to residues 30-110 of SEQ ID NO: 1. In other embodiments, the polypeptide consists of the amino acid sequence corresponding to residues 30-110 of SEQ ID NO: 1. In certain embodiments, the polypeptide is a polypeptide comprising an amino acid sequence that is at least 90%, 95%, or 99% identical to the amino acid sequence corresponding to residues 21-135 of SEQ ID NO: 1. In certain embodiments, the polypeptide comprises the amino acid sequence corresponding to residues 21-135 of SEQ ID NO: 1. In other embodiments, the polypeptide consists of the amino acid sequence corresponding to residues 21-135 of SEQ ID NO: 1.

In some embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. In certain embodiments, the polypeptide consists essentially of the amino acid sequence of SEQ ID NO: 2. In other embodiments, the polypeptide consists of the amino acid sequence of SEQ ID NO: 2.

In some embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In certain embodiments, the polypeptide consists essentially of the amino acid sequence of SEQ ID NO: 3. In other embodiments, the polypeptide consists of the amino acid sequence of SEQ ID NO: 3.

In certain embodiments of the foregoing, the lyophilized polypeptide comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the polypeptide domain and the Fc domain of the immunoglobulin. In certain embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21). In certain embodiments, the linker domain comprises TGGG (SEQ ID NO: 20).

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In certain embodiments, the polypeptide consists essentially of the amino acid sequence of SEQ ID NO: 23. In other embodiments, the polypeptide consists of the amino acid sequence of SEQ ID NO: 23.

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 30. In certain embodiments, the polypeptide consists essentially of the amino acid sequence of SEQ ID NO: 30. In other embodiments, the polypeptide consists of the amino acid sequence of SEQ ID NO: 30.

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41. In certain embodiments, the polypeptide consists essentially of the amino acid sequence of SEQ ID NO: 41. In other embodiments, the polypeptide consists of the amino acid sequence of SEQ ID NO: 41.

In certain embodiments, the lyophilized polypeptide is part of a homodimer protein complex.

In certain embodiments, the polypeptide is glycosylated.

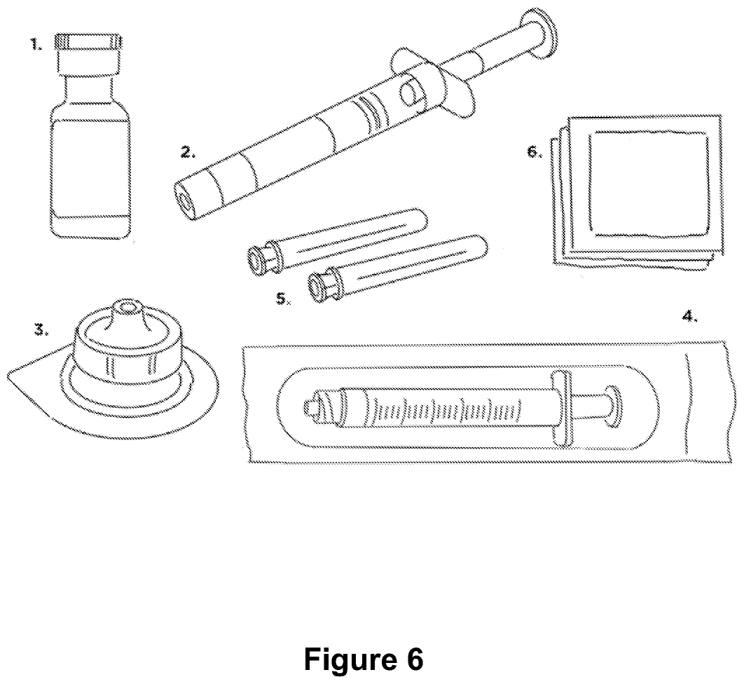

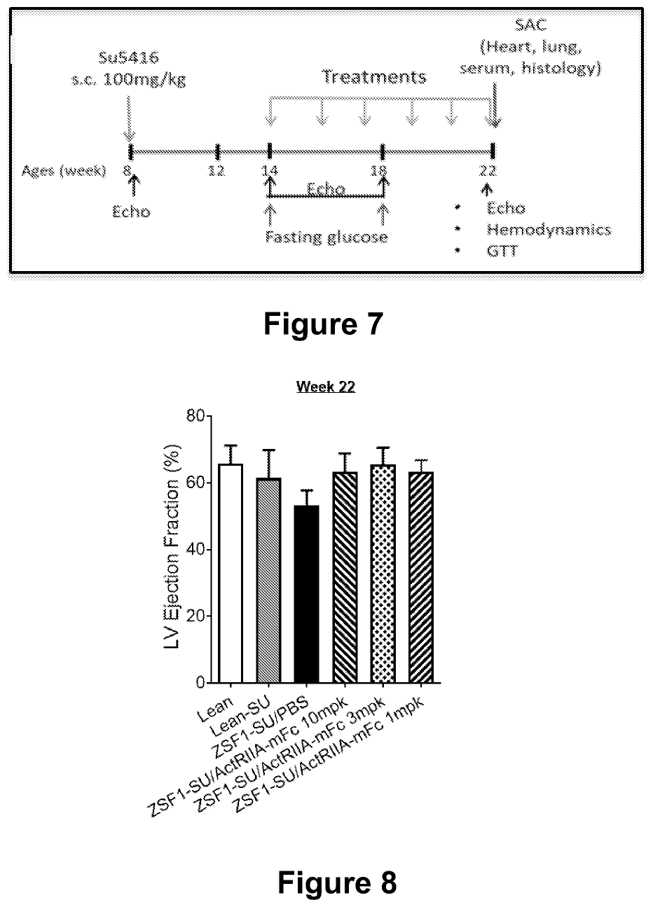

The present disclosure provides a kit comprising a sterile powder comprising a lyophilized polypeptide as disclosed herein and an injection device. In some embodiments of the kits disclosed herein, the sterile powder comprising a lyophilized polypeptide is pre-filled in one or more containers, such as one or more vials [FIG. 6 (1)].

In certain embodiments, the pH range for the sterile powder comprising a lyophilized polypeptide is from 7 to 8. In some embodiments, the sterile powder comprising a lyophilized polypeptide further comprises a buffering agent. In some embodiments, the buffering agent may be added in an amount of at least 10 mM. In some embodiments, the buffering agent may be added in an amount in the range of between about 10 mM to about 200 mM. In some embodiments, the buffering agent comprises citric acid monohydrate and/or trisodium citrate dehydrate.

In some embodiments, the sterile powder comprising a lyophilized polypeptide further comprises a surfactant. In some embodiments, the surfactant comprises a polysorbate. In some embodiments, the surfactant comprises polysorbate 80.

In some embodiments, the sterile powder comprising a lyophilized polypeptide further comprises a lyoprotectant. In some embodiments, the lyoprotectant comprises a sugar, such as disaccharides (e.g., sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized polypeptide in a weight ratio of at least 1:1 lyophilized polypeptide to lyoprotectant. In some embodiments, the sterile powder comprises the lyoprotectant and lyophilized polypeptide in a weight ratio of from 1:1 to 1:10 lyophilized polypeptide to lyoprotectant. In some embodiments, the sterile powder comprises the lyoprotectant and lyophilized polypeptide in a weight ratio of 1:1, 1:2, 1:3, 1:4, 1:5, 1:6, 1:7, 1:8, 1:9, or 1:10 lyophilized polypeptide to lyoprotectant. In some embodiments, the sterile powder comprises the lyoprotectant and lyophilized polypeptide in a weight ratio of 1:6 lyophilized polypeptide to lyoprotectant. In certain embodiments of the foregoing, the sterile powder comprises lyoprotectant in an amount sufficient to stabilize the lyophilized polypeptide.

In certain embodiments of the kits disclosed herein, the injection device comprises a syringe [FIG. 6 (2)]. In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter [FIG. 6 (3)]. In some embodiments, the vial pre-filled with sterile powder comprising a lyophilized polypeptide attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized polypeptide are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized polypeptide reconstitutes the lyophilized polypeptide into a sterile injectable solution. In some embodiments, the lyophilized polypeptide is reconstituted into a sterile injectable solution. In some embodiments, the lyophilized polypeptide is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized polypeptide. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases may be repeated until a desired dose is administered. In certain embodiments, the pump apparatus is coupled to a wearable patch. In certain embodiments, the pump apparatus is a wearable pump apparatus.

The present disclosure provides a kit used for reconstituting a lyophilized polypeptide into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally [FIG. 6 (1, 2, 3, 4, and 5)]. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose.

### 7. Sequences

Human ActRIIB precursor protein sequence
(SEQ ID NO: 39)
MTAPWVALALLWGSLCAGSGRGEAETRECIYYNANWELERTNQSGLERCEGEQDKRLHCY
ASWRNSSGTIELVKKGCWLDDENCYDRQECVATEENPQVYFCCCEGNFCNERFTHLPEAG
GPEVTYEPPPTAPTLLTVLAYSLLPIGGLSLIVLLAFWMYRHRKPPYGHVDIHEDPGPPP
PSPLVGLKPLQLLEIKARGRFGCVWKAQLMNDFVAVKIFPLQDKQSWQSEREIFSTPGMK
HENLLQFIAAEKRGSNLEVELWLITAFHDKGSLTDYLKGNIITWNELCHVAETMSRGLSY
LHEDVPWCRGEGHKPSIAHRDFKSKNVLLKSDLTAVLADFGLAVRFEPGKPPGDTHGQVG
TRRYMAPEVLEGAINFQRDAFLRIDMYAMGLVLWELVSRCKAADGPVDEYMLPFEEEIGQ
HPSLEELQEVVVHKKMRPTIKDHWLKHPGLAQLCVTIEECWDHDAEARLSAGCVEERVSL
IRRSVNGTTSDCLVSLVTSVINVDLPPKESSI
Processed (mature) extracellular ActRIIB polypeptide sequence
(SEQ ID NO: 31)
GRGEAETRECIYYNANWELERTNQSGLERCEGEQDKRLHCYASWRNSSGTIELVKKGCWL
DDENCYDRQECVATEENPQVYFCCCEGNFCNERFTHLPEAGGPEVTYEPPPTAPT
Processed (mature) extracellular ActRIIB polypeptide sequence
with the “tail” deleted (a Δ15 sequence)
(SEQ ID NO: 40)
GRGEAETRECIYYNANWELERTNQSGLERCEGEQDKRLHCYASWRNSSGTIELVKKGCWL
DDENCYDRQECVATEENPQVYFCCCEGNFCNERFTHLPEA

### 8. Exemplification

The disclosure above will be more readily understood by reference to the following examples, which are included merely for purposes of illustration of certain embodiments of the present invention, and are not intended to be limiting.

### Example 1: ActRIIA-Fc Fusion Proteins

A soluble ActRII fusion protein was constructed that has the extracellular domain of human ActRIIA fused to a human or mouse Fc domain with a minimal linker in between. The constructs are referred to as ActRIIA-hFc and ActRIIA-mFc, respectively.

ActRIIA-hFc is shown below as purified from CHO
cell lines (SEQ ID NO: 23):
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K

An additional ActRIIA-hFc lacking the C-terminal lysine is shown below as purified from CHO cell lines (SEQ ID NO: 41):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

The ActRIIA-hFc and ActRIIA-mFc proteins were expressed in CHO cell lines. Three different leader sequences were considered:

(i) Honey bee mellitin (HBML):
(SEQ ID NO: 24)
MKFLVNVALVFMVVYISYIYA
(ii) Tissue plasminogen activator (TPA):
(SEQ ID NO: 25)
MDAMKRGLCCVLLLCGAVFVSP
(iii) Native:
(SEQ ID NO: 26)
MGAAAKLAFAVFLISCSSGA

The selected form employs the TPA leader and has the following unprocessed amino acid sequence:

(SEQ ID NO: 27)
MDAMKRGLCCVLLLCGAVFVSPGAAILGRSETQECLFFNANWEKDRTNQ
TGVEPCYGDKDKRRHCFATWKNISGSIEIVKQGCWLDDINCYDRTDCVE
KKDSPEVYFCCCEGNMCNEKFSYFPEMEVTQPTSNPVTPKPPTGGGTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK

This polypeptide is encoded by the following nucleic acid sequence:

(SEQ ID NO: 28)
ATGGATGCAATGAAGAGAGGGCTCTGCTGTGTGCTGCTGCTGTGTGGAG
CAGTCTTCGTTTCGCCCGGCGCCGCTATACTTGGTAGATCAGAAACTCA
GGAGTGTCTTTTTTTAATGCTAATTGGGAAAAAGACAGAACCAATCAAA
CTGGTGTTGAACCGTGTTATGGTGACAAAGATAAACGGCGGCATTGTTT
TGCTACCTGGAAGAATATTTCTGGTTCCATTGAATAGTGAAACAAGGTT
GTTGGCTGGATGATATCAACTGCTATGACAGGACTGATTGTGTAGAAAA
AAAAGACAGCCCTGAAGTATATTTCTGTTGCTGTGAGGGCAATATGTGT
AATGAAAAGTTTTCTTATTTTCCGGAGATGGAAGTCACACAGCCCACTT
CAAATCCAGTTACACCTAAGCCACCCACCGGTGGTGGAACTCACACATG
CCCACCGTGCCCAGCACCTGAACTCCTGGGGGGACCGTCAGTCTTCCTC
TTCCCCCCAAAACCCAAGGACACCCTCATGATCTCCCGGACCCCTGAGG
TCACATGCGTGGTGGTGGACGTGAGCCACGAAGACCCTGAGGTCAAGTT
CAACTGGTACGTGGACGGCGTGGAGGTGCATAATGCCAAGACAAAGCCG
CGGGAGGAGCAGTACAACAGCACGTACCGTGTGGTCAGCGTCCTCACCG
TCCTGCACCAGGACTGGCTGAATGGCAAGGAGTACAAGTGCAAGGTCTC
CAACAAAGCCCTCCCAGTCCCCATCGAGAAAACCATCTCCAAAGCCAAA
GGGCAGCCCCGAGAACCACAGGTGTACACCCTGCCCCCATCCCGGGAGG
AGATGACCAAGAACCAGGTCAGCCTGACCTGCCTGGTCAAAGGCTTCTA
TCCCAGCGACATCGCCGTGGAGTGGGAGAGCAATGGGCAGCCGGAGAAC
AACTACAAGACCACGCCTCCCGTGCTGGACTCCGACGGCTCCTTCTTCC
TCTATAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAATGAGAATTC

Both ActRIIA-hFc and ActRIIA-mFc were remarkably amenable to recombinant expression. As shown in FIG. 4, the protein was purified as a single, well-defined peak of protein. N-terminal sequencing revealed a single sequence of -ILGRSETQE (SEQ ID NO: 29). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

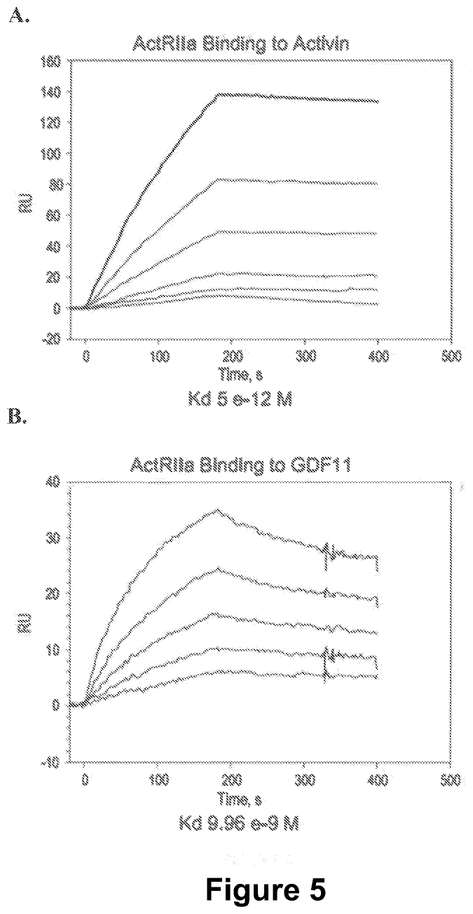

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIG. 5. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 ori/enhancer, CMV promoter), using a tissue plasminogen leader sequence of SEQ ID NO: 25. The protein, purified as described above in Example 1, had a sequence of SEQ ID NO: 23. The Fc portion is a human IgG1 Fc sequence, as shown in SEQ ID NO: 23. Protein analysis reveals that the ActRIIA-hFec fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279(51):53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular domain of ActRIIA). The sequence for such a construct is presented below (Fc portion underlined) (SEQ ID NO: 30):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMTGGGTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPVPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGK

### Example 4: Effects of an ActRIIA-mFc on Group 2 Pulmonary Hypertension in an HFpEF Induced PH Rat Model

The effects of an ActRIIA-mFc fusion protein (ActRIIA-mFc homodimer as described in Example 1) was examined in a rat model of left ventricular diastolic dysfunction (also referred to as HEpEF) Group 2 (subgroup 2.2) pulmonary hypertension (PH). In this model, ZSF1-Lepr^{fa}Lepr^{cp}/Crl rats were challenged with semaxanib to induce HFpEF-PH (Y. C. Lai, Circulation 133, 717-731, 2016).

Forty ZSF1 Lepr^{fa}Lepr^{cp}/Crl male mice (8 wks old) and five lean rats were subcutaneously administered with a single dose of semaxanib (100 mg/kg) at day 0, and five lean rats were included as normal control(“Lean”). Six weeks after semaxanib (SU5416) treatment, Thirty-six ZSF1 Lepr^{fa}Lepr^{cp}/Crl rats were randomized into four groups: i) nine rats were injected subcutaneously with vehicle control (phosphate buffered saline (PBS)), twice weekly for 8 weeks starting from day 42 after semaxanib treatment, “ZSF1-SU/PBS”; a ii) ten rats were injected subcutaneously with ActRIIA-mFc at a dose of 1 mg/kg (mpk) twice weekly for 8 weeks starting from day 42 after semaxanib treatment, “ZSF1-SU/ActRIIA-mFc lmpk”; a iii) nine rats were injected subcutaneously with ActRIIA-mFc at a dose of 3 mg/kg twice weekly for 8 weeks starting from day 42 after semaxanib treatment, “ZSF1-SU/ActRIIA-mFc 3mpk”; and a iv) eight rats were injected subcutaneously with ActRIIA-mFc at a dose of 10 mg/kg twice weekly for 8 weeks starting from day 42 after semaxanib treatment, “ZSF1-SU/ActRIIA-mFc 10mpk”. At the end of the study, echocardiography and pressure-volume catheter were performed to measure left and right ventricular remodeling and functional changes before animals were euthanized for heart and lung collection. Hearts and lungs of each rat were weighed, fixed in 10% formalin, embedded in paraffin, and sectioned for Masson's trichrome stain to assess fibrosis. Serum and urine samples were collected at the end of the study.

Rats were fasted overnight to measure fasting blood glucose levels at week 14 (before treatments started), week 18 (4 weeks after treatments), and week 22, and oral glucose tolerance test was performed at week 22. Blood glucose levels were measured with a glucometer after bleeding tail vein with a 27G needle. To prepare oral glucose tolerance test, 40% glucose stock solution run through a filter to sterilize it. After fasting overnight, rat body weight was measured, and blood glucose level was detected. Then 40% glucose solution was administered via oral gavage according to body weight (2 g/kg). Blood glucose levels were then measured at 30, 60, 90, 120 minutes.

Prior to euthanization, in vivo cardiac function was assessed by transthoracic echocardiography (Acuson P300, 18 MHz linear transducer; Siemens) in lightly anesthetized rats as described (L. Zu, J Mol Cell Cardiol 49, 5-15, 2010). From left ventricle (LV) short axis view, M-mode echocardiogram was acquired to measure interventricular septal thickness at end diastole (IVSd), left ventricular posterior wall thickness at end diastole (LVPWd), left ventricular end diastolic diameter (LVEDD), and left ventricular end systolic diameter (LVESD). Left ventricular mass (LVM) was assessed by the equation: 1.05 [(LVEDD+LVPTD+IVSd)³-LVEDD³]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Pulmonary arterial acceleration time (PAAT), a parameter of right ventricular function, was also measured.

Fourteen weeks after semaxanib treatment, rats were anesthetized with ketamine (100 mg/kg) and xylazine (5 mg/kg) at the end of the experiment to evaluate cardiac and pulmonary hemodynamics. The respiration was supported by a small animal ventilator. Thoracotomy was made through 4-5 intercostal space, and the heart was exposed. A pressure-volume catheter (2.0-Fr, SPR-869, Millar Instruments, Houston, TX, USA) was be inserted into the left ventricle and right ventricle from the apex. Ventricular pressure and volume were calculated with LabChart 7 software. Stroke work, ejection fraction, and cardiac output were derived. After finishing left ventricular measurements, the catheter was advanced to the aorta, arterial blood systolic and diastolic pressure was detected. Then the catheter returned to the left ventricle and changed the direction laterally to enter the left atrium. Similarly, right atrial pressure was measured by moving the catheter from the right ventricle (RV) into atrium. To measure pulmonary arterial pressure, the sternum was cross-sectioned at the second inter-rib space. The right ventricular outflow tract was exposed. A hole was made with 27G needle, and then the catheter was inserted into the right ventricular outflow tract and advanced into the pulmonary artery.

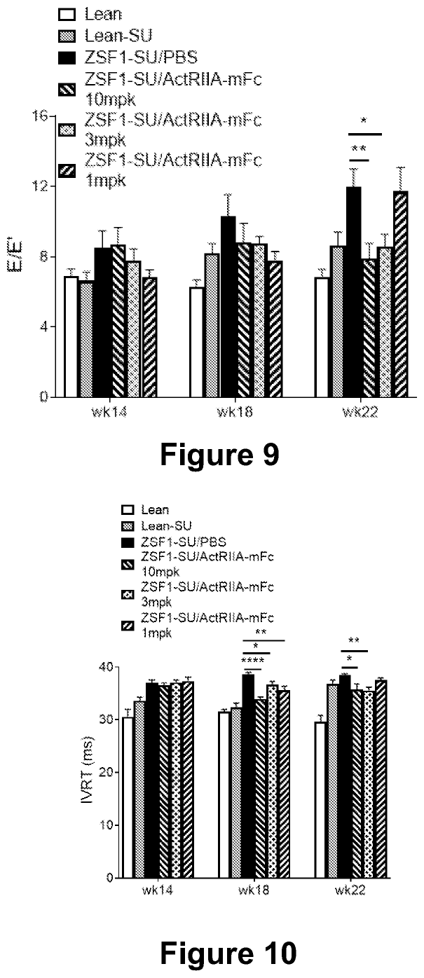

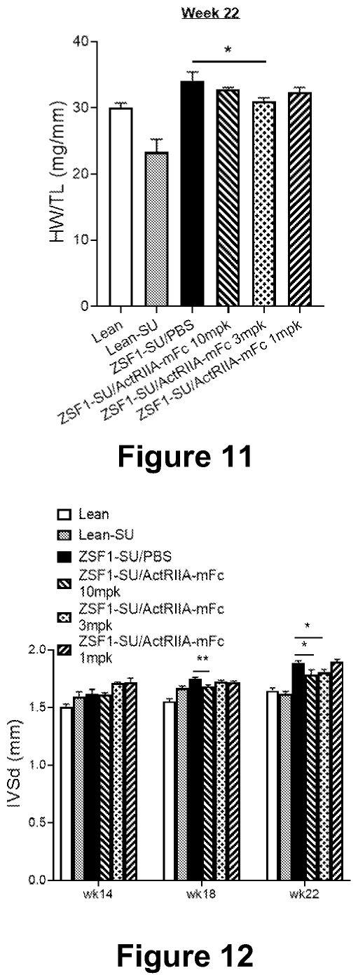

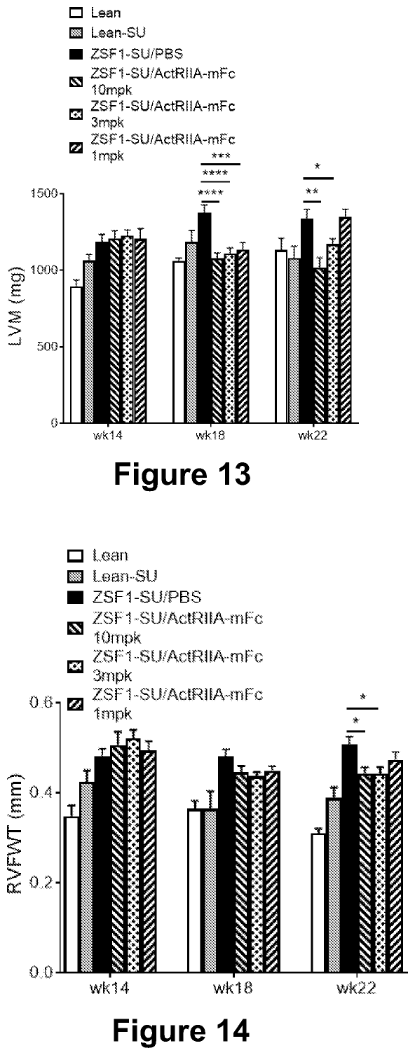

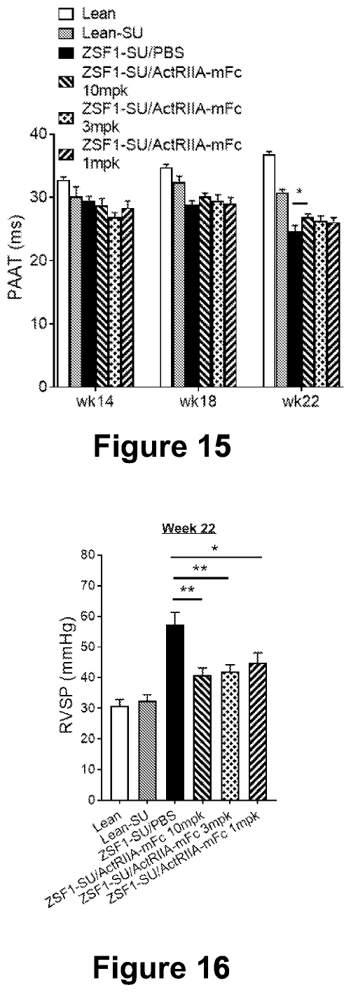

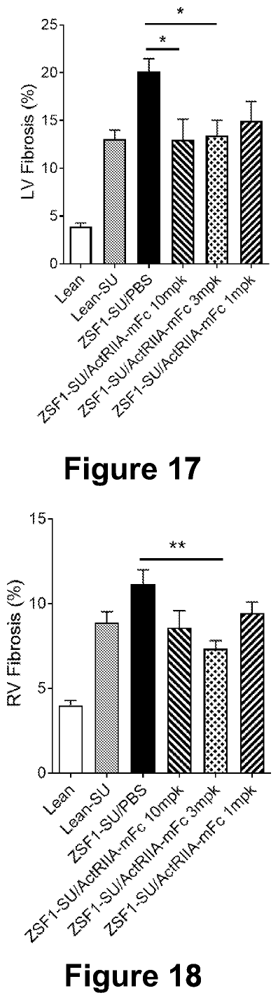

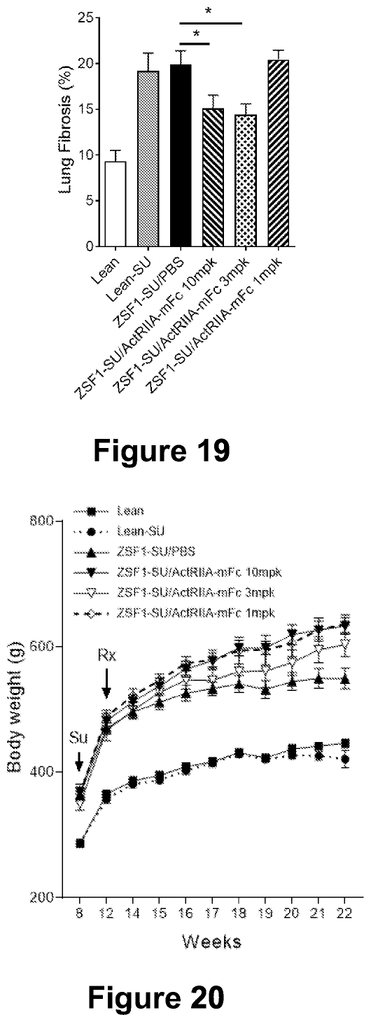

Compared to lean control animals, ZSF1-SU rats in the PBS treatment group (ZSF1-SU/PBS) 14-weeks after semaxanib treatment were observed to have increased heart weight (HW/TL) (FIG. 11), increased IVSd (FIG. 12), and increased LVM (FIG. 13), preserved LV ejection fraction (FIG. 8), increased E/E′ ratio (FIG. 9), increased IVRT (FIG. 10), indicating cardiac hypertrophy and left ventricular diastolic dysfunction. ZSF1 rats also increased right ventricle free wall thickness (RVFWT) (FIG. 14), decreased PAAT (FIG. 15), and increased RVSP (FIG. 16), compared to lean control rats, suggesting pulmonary hypertension and RV remodeling. In addition, increased fibrosis in LV, RV and lung (FIGS. 17-19) was observed in ZSF1-SU/PBS rats.

As shown in the Figures, ActRIIA-mFc treatment (ZSF1-SU/ActRIIA-mFc) relative to PBS treatment (ZSF1-SU/PBS) both at 3 mpk and 10 mpk significantly reduced left heart remodeling (FIGS. 11-13), and reduced E/E′ ratio (FIG. 9), and decreased IVRT (FIG. 10). ActRIIA-mFc treatment (ZSF1-SU/ActRIIA-mFc) relative to PBS treatment (ZSF1-SU/PBS) both at 3 mpk and 10 mpk also significantly reduced elevated RVFWT (FIG. 14), reduced PAAT (FIG. 15), and reduced elevated RVSP (FIG. 16). ActRIIA-mFc treatment (ZSF1-SU/ActRIIA-mFc) relative to PBS treatment (ZSF1-SU/PBS) also significantly reduced fibrosis in LV, RV and lung (FIGS. 17-19).

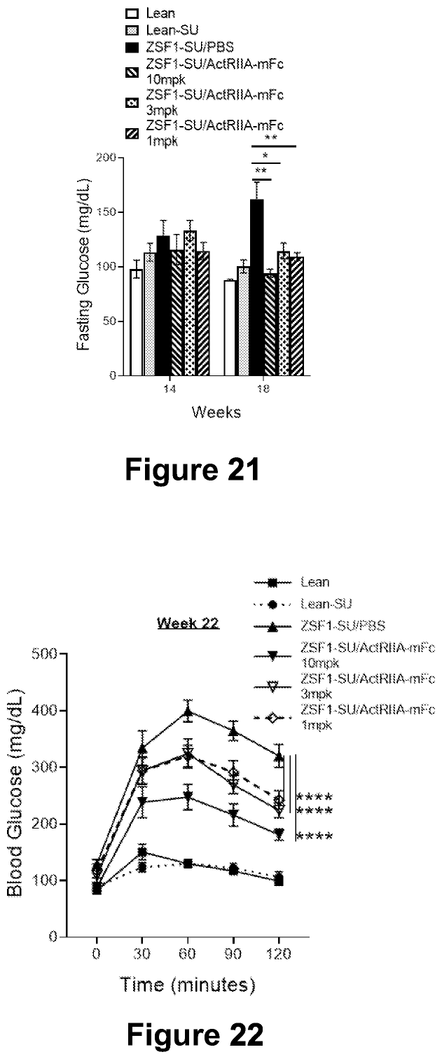

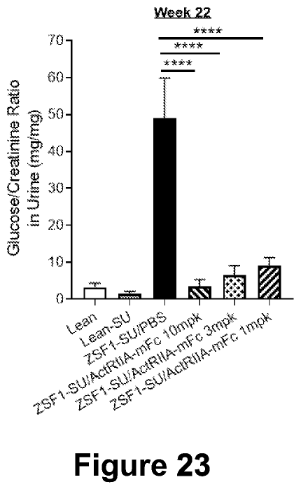

In addition, compared to lean control animals, ZSF1-SU rats in the PBS treatment group (ZSF1-SU/PBS) had elevated fasting blood glucose level and increased glucose level in urine, accompanied by glucose intolerance. ActRIIA-mFc treatment (ZSF1-SU/ActRIIA-mFc) relative to PBS treatment (ZSF1-SU/PBS) at 1 mpk, 3 mpk and 10 mpk significantly reduced fasting blood glucose, decreased glucose level in urine, and improved glucose tolerance (FIGS. 21-23).

Together, these data demonstrate that ActRIIA-mFc is effective in ameliorating various complications of Group 2 PH in a left heart failure-induced PH model (HFpEF-PH). In particular, ActRIIA-mFc had a significant effect in reducing cardiac hypertrophy, improving diastolic function, improving right heart remodeling and function, decreasing pulmonary hypertension, and reducing cardiac and pulmonary remodeling and fibrosis. Furthermore, ActRIIA-mFc had a robust effect in reducing glucose levels and improving glucose tolerance. The data indicate that other ActRII antagonists, particularly ones having activities similar to ActRIIA-mFc, may be useful in the treatment of Group 2 PH, particularly in preventing or reducing the severity various complications of Group 2 PH.

### Example 5: Efficacy Comparison of an ActRIIA-mFc and a SGLT2 Inhibitor on Group 2 Pulmonary Hypertension in an HFpEF Induced PH Rat Model

The effects of an ActRIIA-mFc fusion protein (ActRIIA-mFc homodimer as described in Example 1) was compared with Empagliflozin (SGLT2 inhibitor) in a rat model of left ventricular diastolic dysfunction (also referred to as HEpEF) Group 2 (subgroup 2.2) pulmonary hypertension (PH). In this model, ZSF1-Lepr^{fa}Lepr^{cp}/Crl rats were challenged with semaxanib to induce HFpEF-PH (Y. C. Lai, Circulation 133, 717-731, 2016).

Forty ZSF1 Lepr^{fa}Lepr^{cp}/Crl male rats (8 wks old) and five lean rats were subcutaneously administered with a single dose of semaxanib (100 mg/kg) at day 0, and five lean rats were included as normal control (“Lean”). Six weeks after semaxanib (SU5416) treatment, Thirty-four ZSF1 Lepr^{fa}Lepr^{cp}/Crl rats were randomized into three groups: i) thirteen rats were injected subcutaneously with vehicle control (phosphate buffered saline (PBS)), twice weekly for 8 weeks starting from day 42 after semaxanib treatment, “ZSF1-SU/PBS”; a ii) ten rats were injected subcutaneously with ActRIIA-mFc at a dose of 10 mg/kg (mpk) twice weekly for 8 weeks starting from day 42 after semaxanib treatment, “ZSF1-SU/ActRIIA-mFc”; and a iii) eleven rats were injected subcutaneously with Empagliflozin at a dose of 10 mg/kg once daily for 8 weeks starting from day 42 after semaxanib treatment, “ZSF1-SU/Empa”. At the end of the study, echocardiography and pressure-volume catheter were performed to measure left and right ventricular remodeling and functional changes before animals were euthanized for heart and lung collection. Hearts and lungs of each rat were weighed, fixed in 10% formalin, embedded in paraffin, and sectioned for Masson's trichrome stain to assess fibrosis. Serum samples were collected at the end of the study.

Rats were fasted overnight to measure fasting blood glucose levels at week 14 (before treatments started), and week 22, and oral glucose tolerance test was performed at week 22. Blood glucose levels were measured with a glucometer after bleeding tail vein with a 27G needle. To prepare oral glucose tolerance test, 40% glucose stock solution run through a filter to sterilize it. After fasting overnight, rat body weight was measured, and blood glucose level was detected. Then 40% glucose solution was administered via oral gavage according to body weight (2 g/kg). Blood glucose levels were then measured at 30, 60, 90, 120 minutes.

Prior to euthanization, in vivo cardiac function was assessed by transthoracic echocardiography (Acuson P300, 18 MHz linear transducer; Siemens) in lightly anesthetized rats as described (L. Zu, J Mol Cell Cardiol 49, 5-15, 2010). From left ventricle (LV) short axis view, M-mode echocardiogram was acquired to measure interventricular septal thickness at end diastole (IVSd), left ventricular posterior wall thickness at end diastole (LVPWd), left ventricular end diastolic diameter (LVEDD), and left ventricular end systolic diameter (LVESD). Left ventricular mass (LVM) was assessed by the equation: 1.05 [(LVEDD+LVPTD+lVSd)³-LVEDD³]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Pulmonary arterial acceleration time (PAAT), a parameter of right ventricular function, was also measured.

Fourteen weeks after semaxanib treatment, rats were anesthetized with ketamine (100 mg/kg) and xylazine (5 mg/kg) at the end of the experiment to evaluate cardiac and pulmonary hemodynamics. The respiration was supported by a small animal ventilator. Thoracotomy was made through 4-5 intercostal space, and the heart was exposed. A pressure-volume catheter (2,0-Fr, SPR-869, Millar Instruments, Houston, TX, USA) was be inserted into the left ventricle (LV) and right ventricle (RV) from the apex. Ventricular pressure and volume were calculated with LabChart 7 software. Stroke work, ejection fraction, and cardiac output were derived. After finishing left ventricular measurements, the catheter was advanced to the aorta, arterial blood systolic and diastolic pressure was detected. Then the catheter returned to the left ventricle and changed the direction laterally to enter the left atrium. Similarly, right atrial pressure was measured by moving the catheter from the right ventricle into atrium. To measure pulmonary arterial pressure, the sternum was cross-sectioned at the second inter-rib space. The right ventricular outflow tract was exposed. A hole was made with 27G needle, and then the catheter was inserted into the right ventricular outflow tract and advanced into the pulmonary artery.

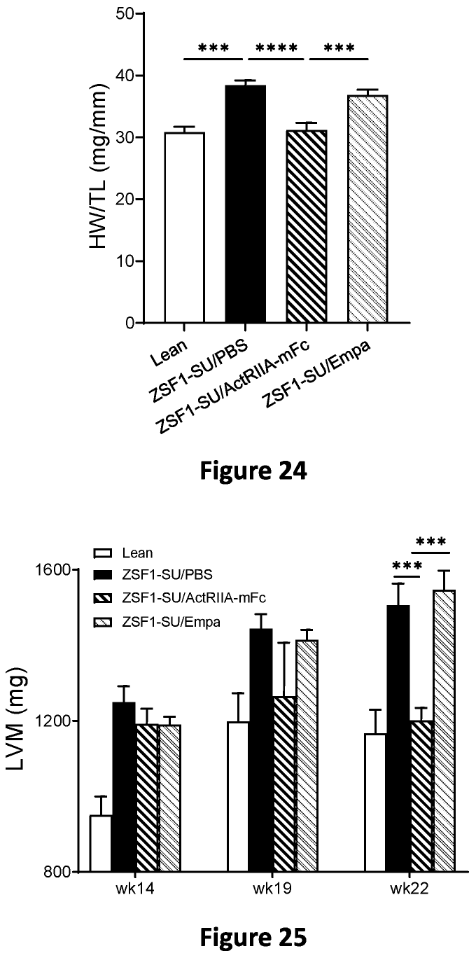

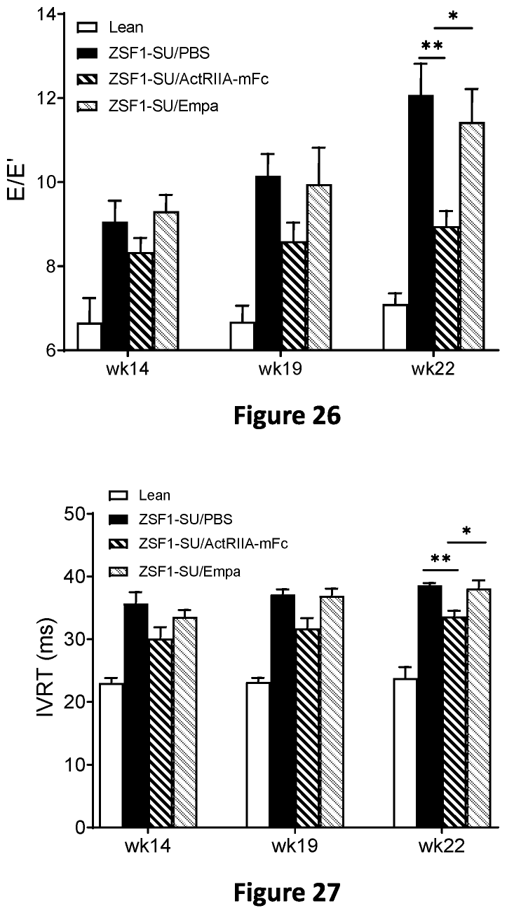

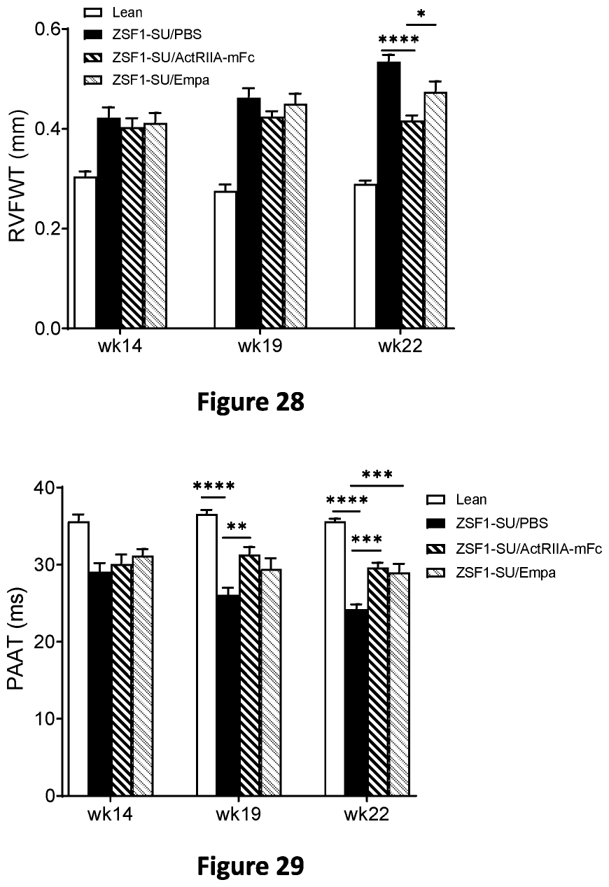

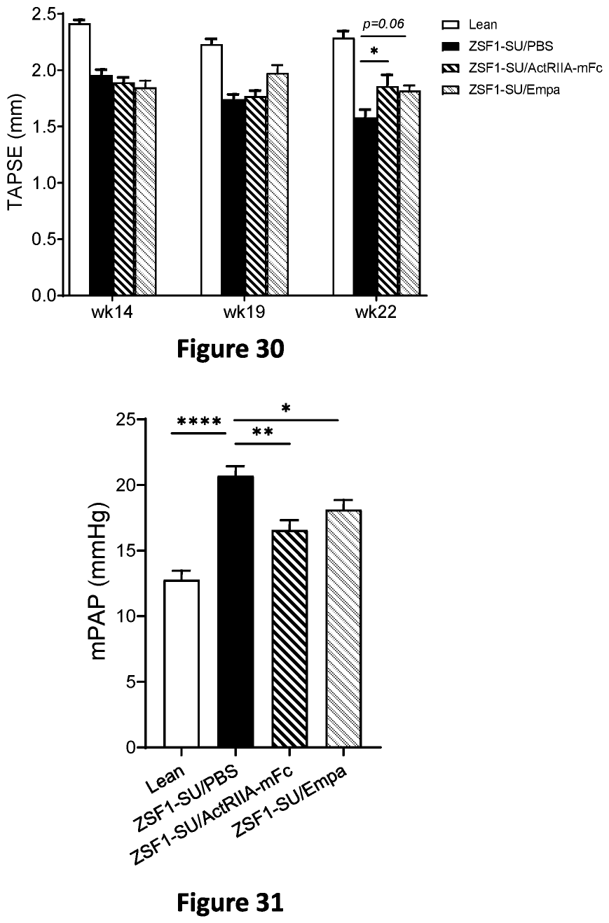

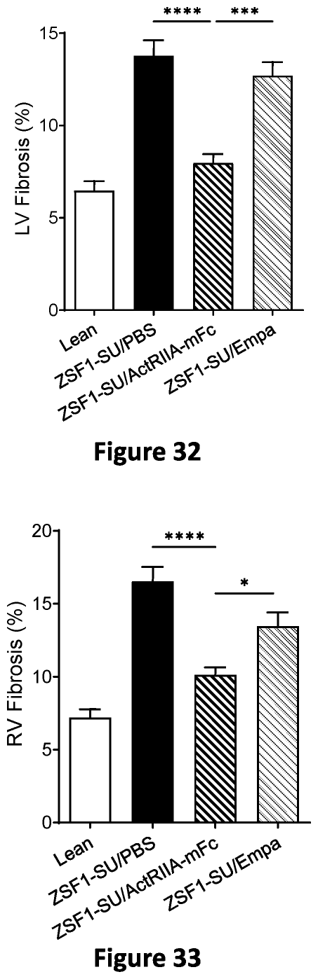

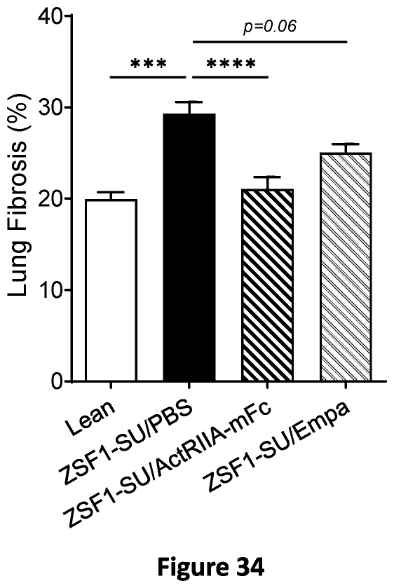

Compared to lean control animals, ZSF1-SU rats in the PBS treatment group (ZSF1-SU/PBS) 14-weeks after semaxanib treatment were observed to have increased heart weight (HW/TL) (FIG. 24), and increased LVM (FIG. 25), increased E/E′ ratio (FIG. 26), increased IVRT (FIG. 27), indicating cardiac hypertrophy and left ventricular diastolic dysfunction. ZSF1 rats also increased right ventricle free wall thickness (RVFWT) (FIG. 28), decreased PAAT (FIG. 29), decreased TAPSE (FIG. 30), and increased mean pulmonary arterial pressure (mPAP) (FIG. 31), compared to lean control rats, suggesting pulmonary hypertension and RV remodeling. In addition, increased fibrosis in LV, RV and lung (FIGS. 32-34) was observed in ZSF1-SU/PBS rats.

As shown in the Figures, ActRIIA-mFc treatment (ZSF1-SU/ActRIIA-mFc) relative to either PBS treatment (ZSF1-SU/PBS) or Empagliflozin treatment (ZSF1-SU/Empa) significantly reduced left heart remodeling (FIGS. 24-26), and reduced E/E′ ratio (FIG. 26), and decreased IVRT (FIG. 27). Both ActRIIA-mFc treatment (ZSF1-SU/ActRIIA-mFc) and Empagliflozin treatment (ZSF1-SU/Empa) relative to PBS treatment (ZSF1-SU/PBS) significantly reduced elevated RVFWT (FIG. 28), increased reduced PAAT (FIG. 29), increased reduced TAPSE (FIG. 30), and reduced elevated mPAP (FIG. 31). However, ActRIIA-mFc treatment (ZSF1-SU/ActRIIA-mFc) generally appeared to be more potent modifier of these parameters (e.g., RVFWT, TAPSE, PAAT, and mPAP) compared to Empagliflozin treatment (ZSF1-SU/Empa) treatment. ActRIIA-mFc treatment (ZSF1-SU/ActRIIA-mFc) relative to PBS treatment (ZSF1-SU/PBS) significantly reduced fibrosis in LV, RV and lung (FIGS. 32-34). In comparison, Empagliflozin treatment (ZSF1-SU/Empa) had only modest, if any, effects on reducing fibrosis in LV and RV (FIG. 32-34).

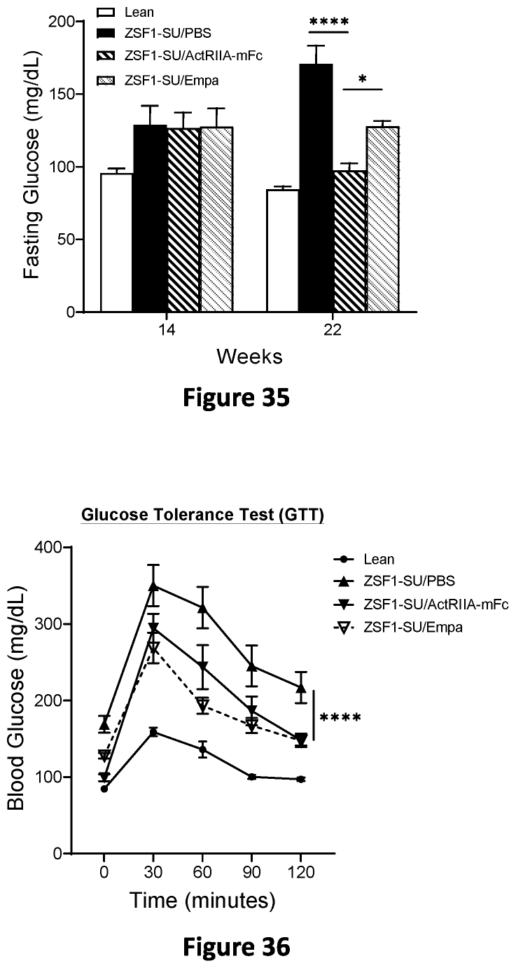

In addition, compared to lean control animals, ZSF1-SU rats in the PBS treatment group (ZSF1-SU/PBS) had elevated fasting blood glucose level, accompanied by glucose intolerance. ActRIIA-mFc treatment (ZSF1-SU/ActRIIA-mFc) relative to PBS treatment (ZSF1-SU/PBS) at 10 mpk significantly reduced fasting blood glucose and improved glucose tolerance at week 22 (FIGS. 35 and 36). In comparison, Empagliflozin treatment (ZSF1-SU/Empa) had only modest effects on reduced fasting blood glucose and improving glucose tolerance at week 22 (FIGS. 35 and 36).

Together, these data demonstrate that ActRIIA-mFc is effective in ameliorating various complications of Group 2 PH in a left heart failure-induced PH model (HFpEF-PH). In particular, ActRIIA-mFc had a significant effect in reducing cardiac hypertrophy, improving diastolic function, improving right heart remodeling and function, decreasing pulmonary hypertension, and reducing cardiac and pulmonary remodeling and fibrosis, together with the reduction of fasting blood glucose and the improvement of glucose tolerance. Furthermore, ActRIIA-mFc is more effective than Empagliflozin in treating diastolic function, left heart remodeling, fibrosis, and glucose levels/tolerance. The data indicate that other ActRII antagonists, particularly ones having activities similar to ActRIIA-mFc, may be useful in the treatment of Group 2 PH, particularly in preventing or reducing the severity various complications of Group 2 PH.

1. method of treating hyperglycemia associated with pulmonary hypertension, comprising administering to a patient in need thereof an effective amount of an ActRII polypeptide comprising an amino acid sequence that is at least 85% identical to an amino acid sequence that begins at any one of amino acids 110-135 of SEQ ID NO: 1.

2-7. (canceled)

8. The method of claim 1, wherein the pulmonary hypertension is combined post- and pre-capillary pulmonary hypertension (CpcPH).

9. The method of claim 1, wherein the pulmonary hypertension is Group 2 pulmonary hypertension.

10. (canceled)

11. The method of claim 1, wherein administration of the polypeptide results in a decrease in fasting glucose levels in the patient to levels less than 100 mg/dL.

12. The method of claim 1, wherein administration of the polypeptide results in a decrease in fasting glucose levels in the patient by at least 15%.

13. The method of claim 1, wherein administration of the polypeptide decreases blood glucose levels two hours after eating by at least 15% or by at least 50 mg/dL.

14. (canceled)

15. The method of claim 1, wherein administration of the polypeptide decreases the glucose/creatinine ratio in the patient's urine to less than 15 mg/mg, by at least 30 mg/mg, or by at least 50%.

16-42. (canceled)

43. The method of claim 1, wherein the patient further has a condition selected from the group consisting of prediabetes, diabetes mellitus, type 1 diabetes mellitus, and type 2 diabetes mellitus.

44-55. (canceled)

56. The method of claim 1, wherein the ActRII polypeptide an amino acid sequence that is at least 90% identical to the sequence of amino acids corresponding to residues 30-110 of SEQ ID NO: 1.

57. The method of claim 1, wherein the ActRII polypeptide comprises an amino acid sequence that is at least 85% identical to the amino acid sequence SEQ ID NO: 2.

58. The method of claim 1, wherein the ActRII polypeptide comprises an amino acid sequence that is at least 85% identical to the amino acid sequence of SEQ ID NO: 3.

59. The method of claim 56, wherein the ActRII polypeptide is a fusion protein further comprising an Fc domain of an immunoglobulin.

60. The method of claim 59, wherein the Fe domain of the immunoglobulin is an Fe domain of an IgG1 immunoglobulin.

61. The method of claim 59, wherein the Fc fusion protein further comprises a linker domain positioned between the ActRII polypeptide domain and the Fe domain of the immunoglobulin.

62. The method of claim 61, wherein the linker domain is selected from the group consisting of: TGGG (SEQ ID NO: 20), TGGGG (SEQ ID NO: 18), SGGGG (SEQ ID NO: 22), GGGGS (SEQ ID NO: 22), GGG (SEQ ID NO: 16), GGGG (SEQ ID NO: 17), and SGGG (SEQ ID NO: 21).

63. The method of claim 1, wherein the ActRII polypeptide comprises an amino acid sequence that is at least 90% identical to the amino acid sequence of SEQ ID NO: 23.

64. (canceled)

65. The method of claim 1, wherein the ActRII polypeptide comprises an amino acid sequence that is at least 90% identical to the amino acid sequence of SEQ ID NO: 41.

66-68. (canceled)

69. The method of claim 1, wherein the ActRII polypeptide binds to one or more ligands selected from the group consisting of: activin A, activin B, and GDF11.

70. The method of claim 1, wherein the ActRII polypeptide is administered at a dose between 0.1 mg/kg and 2.0 mg/kg.

71-72. (canceled)

73. The method of claim 1, wherein the ActRII polypeptide is administered to the patient every week, every two weeks, every three weeks, or every four weeks.

74-75. (canceled)
